(19) United States
(12) Patent Application Publication
(10) Pub. No.: US 2023/0219886 A1
(43) Pub. Date: Jul. 13, 2023

(54) NOVEL SELENO-NSAID ANALOGS AND USES THEREOF

(71) Applicants: María del Carmen SANMARTIN GRIJALBA, Pamplona, Navarra (ES); Daniel PLANO AMATRIAIN, Pamplona, Navarra (ES); Ana Carolina RUBERTE SÁNCHEZ, Pamplona, Navarra (ES); THE PENN STATE RESEARCH FOUNDATION, University Park, PA (US)

(72) Inventors: Arun K. Sharma, Hummelstown, PA (US); Shantu G. Amin, Scottsdale, AZ (US); Mohammad Asif Raza, Hershey, PA (US); Ana Carolina Ruberte Sánchez, Pamplona, Navarra (ES); Daniel Plano Amatriain, Pamplona, Navarra (ES); María del Carmen Sanmartín Grijalba, Pamplona, Navarra (ES)

(21) Appl. No.: 18/007,724

(22) PCT Filed: Jun. 2, 2021
(86) PCT No.: PCT/US2021/035474

§ 371 Date: Dec. 1, 2022

Related U.S. Application Data
(60) Provisional application No. 63/033,523, filed on Jun. 2, 2020.

(51) Int. Cl. C07C 391/00; A61P 35/00; C07D 209/28

(52) U.S. Cl. CPC C07C 391/00; A61P 35/00; C07D 209/28

(57) ABSTRACT

The present invention relates to seleno-NSAID compounds, compositions comprising such compounds, and methods of use. In particular, provided herein are compounds, pharmaceutical compositions, and methods of using of the same for treating cancer and for reducing or inhibiting cancer cell growth in a subject having cancer.

### CROSS-REFERENCE TO RELATED APPLICATIONS

This application claims the benefit of priority under 35 U.S.C. § 119(e) to U.S. Provisional Application No. 63/033,523, filed Jun. 2, 2020, the content of which is incorporated herein by reference in its entirety.

### BACKGROUND

Non-steroidal anti-inflammatory drugs (NSAIDs) are therapeutic agents that simultaneously stimulate immune system and inhibit tumor-associated inflammation. To date, several epidemiological, clinical and preclinical studies have supported that daily dosing of NSAIDs, such as aspirin (ASA), indomethacin (Inn) and sulindac (Sulin), are associated with reduced risk for certain cancers, including colorectal and lung cancers. Meta-analyses and systematic reviews suggest that the use of ASA and other NSAIDs is related with decreased incidence of colon adenomas, as well as cell growth inhibition of colorectal cancer cells (CRC), and metastatic CRC in patients with PIK3CA-mutants cancers. Additionally, ASA, Inn, Sulin, Naproxen (Nap) and related NSAIDs have showed promising anti-neoplastic effects in various cancers. In this context, these effects may be due to several mechanisms of action, including activation of nuclear factor kappa B (NF-kB); cyclooxygenases (COXs) inhibition and reduction of prostaglandins; reduction of VEGF expression and oxidative stress; regulation of the catabolism and export of intracellular polyamines; suppression of beta-catenin-dependent transcription and disruption of autophagic flux by disturbing the normal functioning of lysosomes. Additionally, they induced apoptosis by a series of biochemical events, including intracellular ceramide generation, dephosphorylation of Akt and release of Smac into the cytosol. Furthermore, there are strong genetic evidences that TGFβ signaling and/or its effectors participate in NSAID-dependent anti-neoplastic processes. Interestingly, the administration of NSAIDs post-operatively was related with longer overall and progression-free survival in non-small cell lung cancer patients with post-operative fever.

### SUMMARY OF THE DISCLOSURE

The present disclosure provides seleno-NSAID compounds, compositions comprising such compounds, and methods of use.

In a first aspect, provided herein are compounds of formula (I), or pharmaceutically acceptable salts thereof:

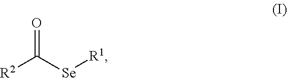

wherein R¹ is selected from alkyl (e.g., branched or unbranched C₁-C₁₂ alkyl such as methyl or ethyl), aryl (e.g., phenyl), or (aryl)alkyl (e.g., -methyl-aryl such as benzyl);
wherein R² is selected from aryl (e.g., phenyl), (aryl)alkyl (e.g., -methyl-naphthyl or -methyl-indenyl), heteroaryl (e.g., indolyl), and (heteroaryl)alkyl (e.g., -methyl-indolyl), and R² optionally is substituted with one or more R^{A}; and
wherein R^{A} is selected from alkyl (e.g., branched or unbranched C₁-C₁₂ alkyl such as methyl or ethyl), alkoxy (e.g., methoxy), hydroxyl, —O—C(O)H, —O—C(O)—CH₃, halogen (e.g., —Cl or —F), phenyl optionally substituted with one or more halogens, N-anilino optionally substituted with alkyl, —C(O)-phenyl optionally substituted with halogen, —CH═CH— phenyl optionally substituted with —S(O)—CH₃.

In another aspect, provided herein are pharmaceutical compositions comprising the compounds or pharmaceutically acceptable salts thereof described herein.

In a further aspect, provided herein is a method of treating cancer in a subject in need thereof. The method can comprise or consist essentially of administering the compounds or pharmaceutically acceptable salts described herein to the subject. Administration of the compounds or pharmaceutically acceptable salts described herein can be in combination with one or more additional anti-cancer therapies. The cancer to be treated by the method provided herein can be selected from the group consisting of pancreatic ductal adenocarcinoma, melanoma, prostate cancer, breast cancer, renal cancer, and any combination thereof.

In a further aspect, provided herein are compositions comprising the compounds, or pharmaceutically acceptable salts thereof, described herein for use in the inhibition of tumor growth and treatment of cancer.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents and alternatives. Reference should therefore be made to the claims recited herein for interpreting the scope of the invention.

### BRIEF DESCRIPTION OF THE DRAWINGS

FIGS. 1A-1B present structures of (A) phospho-, NO- and H₂S-releasing NSAIDs and (B) methylseleno precursors.

FIG. 2 presents structural design of novel methylseleno-NSAIDs (series 1), along with ethylseleno- and benzylseleno-NSAID analogs (series 2).

FIGS. 3A-3I present dose-response curves obtained for 1a (A-C), 5a (D-F) and 6a (G-I), NSAIDs (ASA, Inn and Sulin, respectively), MSA and combination of MSA+ASA in HT-29 cell line. (A-C) HT-29 cell survival after treating the cells for 24 hours (h) (A), 48 h (B) and 72 h (C) with 1a, ASA, MSA and combination of MSA+ASA. (D-F) HT-29 cell survival after treating the cells for 24 h (D), 48 h (E) and 72 h (F) with 5a, Inn, MSA and combination of MSA+Inn. (G-I) HT-29 cell survival after treating the cells for 24 h (G), 48 h (H) and 72 h (I) with 6a, Sulin, MSA and combination of MSA+Sulin.

FIGS. 4A-4I present dose-response curves obtained for 1a (A-C), 5a (D-F) and 6a (G-I), NSAIDs (ASA, Inn and Sulin, respectively), MSA and combination of MSA+ASA in HCT-116 cell line. (A-C) HCT-116 cell survival after treating the cells for 24 h (A), 48 h (B) and 72 h (C) with 1a, ASA, MSA and combination of MSA+ASA. (D-F) HCT-116 cell survival after treating the cells for 24 h (D), 48 h (E) and 72 h (F) with 5a, Inn, MSA and combination of MSA+Inn. (G-I) HCT-116 cell survival after treating the cells for 24 h (G), 48 h (H) and 72 h (I) with 6a, Sulin, MSA and combination of MSA+Sulin.

FIGS. 5A-5I present dose-response curves obtained for 1a (A-C), 5a (D-F) and 6a (G-I), NSAIDs (ASA, Inn and Sulin, respectively), MSA and combination of MSA+ASA in RKO cell line. (A-C) RKO cell survival after treating the cells for 24 h (A), 48 h (B) and 72 h (C) with 1a, ASA, MSA and combination of MSA+ASA. (D-F) RKO cell survival after treating the cells for 24 h (D), 48 h (E) and 72 h (F) with 5a, Inn, MSA and combination of MSA+Inn. (G-I) RKO cell survival after treating the cells for 24 h (G), 48 h (H) and 72 h (I) with 6a, Sulin, MSA and combination of MSA+Sulin.

FIGS. 6A-61 present dose-response curves obtained for 1a (A-C), 5a (D-F) and 6a (G-I), NSAIDs (ASA, Inn and Sulin, respectively), MSA and combination of MSA+ASA in Caco-2 cell line. (A-C) Caco-2 cell survival after treating the cells for 24 h (A), 48 h (B) and 72 h (C) with 1a, ASA, MSA and combination of MSA+ASA. (D-F) Caco-2 cell survival after treating the cells for 24 h (D), 48 h (E) and 72 h (F) with 5a, Inn, MSA and combination of MSA+Inn. (G-I) Caco-2 cell survival after treating the cells for 24 h (G), 48 h (H) and 72 h (I) with 6a, Sulin, MSA and combination of MSA+Sulin.

FIGS. 7A-7B demonstrate that compound 1a and 6a inhibited the CRC cell cycle. (A) HT-29 were serum starved for 72 h, followed by treatment with 1a and 6a (1, 2.5, 5 and 10 μM) and camptothecin (10 μM) in media with serum. Cell cycle phase distribution was determined at 24 h by flow-cytometry. These results are grouped in Bar diagram representing the distribution of cells in different phases of the cell cycle. (B) HT-29 cells were treated with compounds 1a and 6a (2 and 3 μM, respectively) for the indicated time durations (0, 12 and 24 h). Whole cell lysates were subjected to the expression analysis by Western blotting using respective antibodies. (3-actin was used as an endogenous control.

FIGS. 8A-8B demonstrate that compounds 1a and 6a induced apoptosis in CRC cells. (A) and (B), Annexin V & Dead cell and Caspase 3/7 Assays led to the distribution of the cells into four different cell population after treatment. Healthy cells (Annexin-V negative and caspase 3/7 and 7-ADD negative (lower right quadrant)), early apoptotic cells (positive for Annexin-V and caspase 3/7 and negative for 7-ADD (lower right corner)), late apoptotic/dead cells (both Annexin V and caspase 3/7 and 7-ADD positive (upper right quadrant)), and necrotic cells (only 7-ADD positive (Upper left quadrant)).

FIG. 9 presents dose-response curves for 6a derivative on NCI-60 human cell lines after 48 hours (h) of treatment.

FIG. 10 demonstrates in vivo efficacy of methylseleno-sulindac analog 6a against pancreatic ductal adenocarcinoma in a subcutaneous xenograft nude mouse model using Panc-1 cells.

### DETAILED DESCRIPTION

The present invention is based at least in part on the development of compounds referred to as Selenium-Nonsteroidal anti-inflammatory drugs or “Se-NSAIDs.” In one embodiment, the disclosed compounds were developed by incorporating methylseleno-, ethylseleno- and benzylseleno-moieties into structures of NSAIDs. As described herein, the methylseleno-, ethylseleno-, and benzylseleno-NSAID analogs of this disclosure exhibit significantly increased cytotoxic potency relative to their parent NSAID scaffolds or parent moieties, as well as unique cytotoxicity activity profiles towards a National Cancer Institute's (NCI) Developmental Therapeutics Program (DTP) panel of 60 cancer cell lines.

Accordingly, in a first aspect, provided herein is a compound of formula (I), or a pharmaceutically acceptable salt thereof:

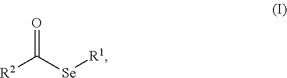

wherein R¹ is selected from a branched or unbranched C₁-C₁₂ alkyl, or a branched or unbranched C₆-C₁₂ (aryl)alkyl;

wherein R² is selected from a substituted or unsubstituted aryl, a substituted or unsubstituted C₆-C₁₂ heteroaryl, a branched or unbranched, substituted or unsubstituted C₆-C₁₂ (heteroaryl)alkyl or a branched or unbranched, substituted or unsubstituted C₆-C₁₂ (aryl)alkyl, wherein the R² optionally is substituted with one or more R^{A};

wherein R^{A} is selected from halo, —OR^{B}, a branched or unbranched C₁-C₁₂ alkyl, aryl, —NHR^{C}, —C(O)R^{E}, a branched or unbranched, substituted or unsubstituted C₂-C₁₂ (aryl)alkenyl, wherein the aryl and (aryl)alkenyl are each optionally substituted with R^{F};

wherein R^{B} is selected from hydrogen, C₁-C₆ alkyl, or —C(O)—H or —C(O)-alkyl (e.g. C₁-C₆ alkyl);

wherein R^{C} and R^{E} are each independently selected from substituted or unsubstituted C₆-C₁₂ aryl, wherein the aryl is optionally substituted with R^{H};

wherein R^{F} and R^{H}, are each independently selected from halo, a branched or unbranched C₁-C₁₂ alkyl, or —S(O)-alkyl (e.g. C₁-C₆ alkyl).

In some cases, R¹ is selected from the branched or unbranched, substituted or unsubstituted C₁-C₁₂ alkyl.

In some cases, R¹ is selected from the branched or unbranched, substituted or unsubstituted C₆-C₁₂ (aryl)alkyl, or substituted or unsubstituted aryl.

In some cases, R¹ is selected from phenyl or benzyl.

In some cases, R² is selected from substituted or unsubstituted aryl, the branched or unbranched, substituted or unsubstituted C₆-C₁₂ (heteroaryl)alky or the branched or unbranched, substituted or unsubstituted C₆-C₁₂ (aryl)alkyl. For example, R² may be selected from phenyl, benzyl, naphthalene, methylnaphthalene, indolyl, 1-methylindolyl, indenyl, or 1-methyl-1H-indenyl, wherein each R² is substituted with one or more R^{A} groups.

In some cases, R^{A} is selected from halo, hydroxyl, —OR^{B}, —NHR^{C}, C₁-C₆ alkyl, —C(O)R^{E}, phenyl, or benzyl, wherein the phenyl and the benzyl are each optionally substituted with R^{F}.

In some cases, R^{B} is selected from —C(O)C₁-C₆ alkyl or C₁-C₆ alkyl.

In some cases, R^{C} and R^{E} are each independently a phenyl substituted with R^{H}.

In some cases, R^{F} and R^{H}, are each independently selected from halo, C₁-C₆ alkyl, or —S(O)Me. In another aspect, provided herein is a compound of formula (II), or a pharmaceutically acceptable salt thereof:

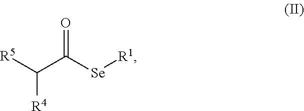

wherein R⁴ is selected from a substituted or unsubstituted aryl or a substituted or unsubstituted C₆-C₁₂ heteroaryl, wherein the aryl and the heteroaryl, are each optionally substituted with Rⁱ,

wherein Rⁱ is selected from halo, —OR^{B}, C₁-C₆ alkyl, C(O)R^{E}, or benzyl, wherein the benzyl is optionally substituted with R^{F},

wherein R^{B} is selected from C₁-C₆ alkyl,

wherein R^{E} is a phenyl substituted with R^{H},

wherein R^{F} and R^{H}, are each independently selected from halo, C₁-C₆ alkyl, or —S(O)Me;

wherein R⁵ is selected from hydrogen or C₁-C₆ alkyl.

In another aspect, provided herein is a compound, or a pharmaceutically acceptable salt thereof, selected from the group consisting of:

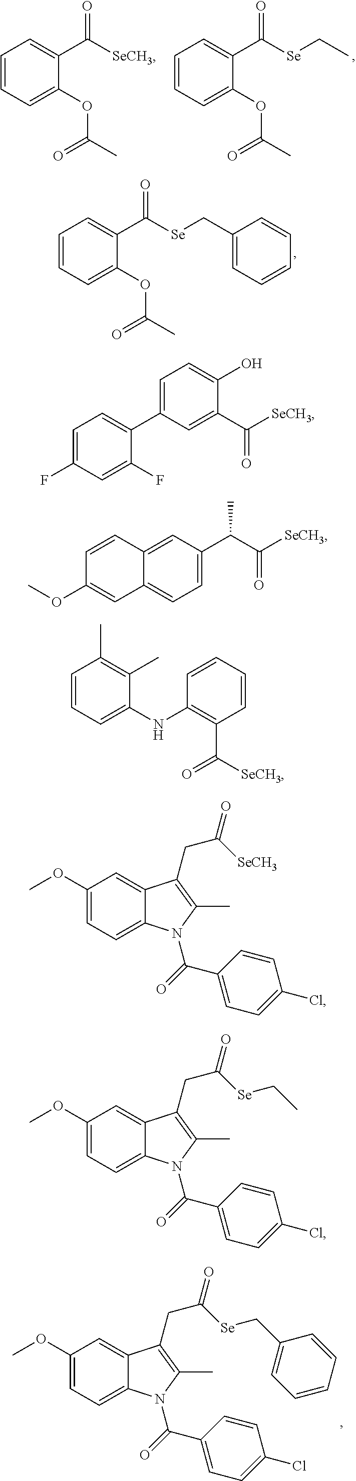

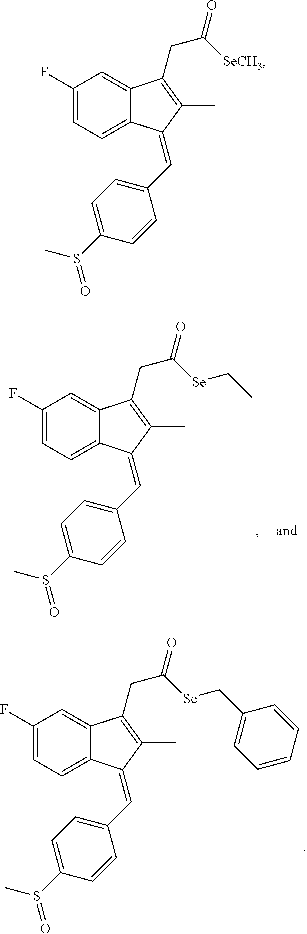

In some cases, the compound is a methylseleno analog selected from those identified herein is as compound 1a, 5a, and 6a:

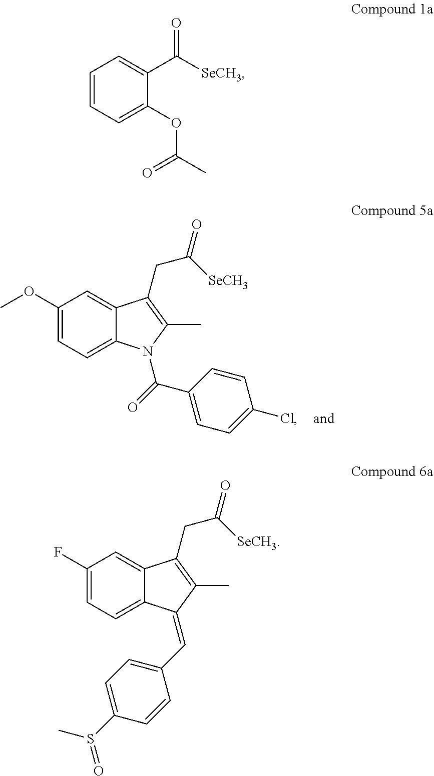

Three methylseleno analogs (1a, 5a and 6a) exhibited significantly increased cytotoxic potency as compared to methylselenilic acid and parent NSAID scaffolds (aspirin, indomethacin and sulindac, respectively) alone or in combination in colorectal cancer (CRC) cells. Interestingly, these analogs showed completely different activity profiles towards a National Cancer Institute's (NCI) Developmental Therapeutics Program (DTP) panel of 60 cancer cell lines.

Methylseleno-sulindac analog 6a, also referred to herein as compound 6a, exhibited potent cytotoxicity against the six most drug-resistant cancer cell lines of the National Cancer Institute's (NCI) Developmental Therapeutics Program (DTP) panel, with cytotoxic parameter values of 47, 410 and 3900 nM for GI50, TGI and LC50, respectively, in the most resistant ovarian cancer cell line (OVCAR-5). In addition, compound 6a (4 mg/k, 3×wk) significantly reduced subcutaneous tumor growth in a pancreatic cancer mouse xenograft.

In some cases, the non-steroidal anti-inflammatory drug (NSAID) is selected from the group consisting of a salicylate compound, a propionic acid compound, a selective COX-2 inhibitor, and an acetic acid compound. Salicylate compounds include, without limitation, acetylsalicylic acid (aspirin), diflunisal, and salsalate. Propionic acid compounds include, without limitation, ibuprofen, naproxen, fenoprofen, ketoprofen, flurbiprofen, oxaprozin, and loxoprofen. Selected COX-2 inhibitor compounds include, without limitation, COXIBs such as celecoxib and rofecoxib. Acetic acid compounds include, without limitation, indomethacin, sulindac, etodolac, ketorolac, diclofenac, and nabumetone.

In some cases, a compound of this disclosure, or a pharmaceutically acceptable salt thereof, is combined with at least one pharmaceutically acceptable carrier, diluent, or excipient to form a composition. As used herein, the phrase “pharmaceutically acceptable” refers to those agents, compounds, materials, compositions, and/or dosage forms which are, within the scope of sound medical judgment, suitable for use in contact with the tissues of humans and animals without excessive toxicity, irritation, allergic response, or other problems or complications, commensurate with a reasonable benefit/risk ratio. Compositions provided herein can comprise at least one pharmaceutically acceptable diluent, excipient, or carrier. As used herein, “diluent, excipient, or carrier” refers to any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The compounds of the invention are advantageously used in the inhibition of tumor growth and treatment of cancer. Accordingly, in another aspect, provided herein are methods of administering a compound or pharmaceutically acceptable salt thereof to a subject to treat cancer. As used herein the term “cancer” refers to a physical condition in mammals that is typically characterized by a group of cells that display uncontrolled growth (division beyond the normal limits), invasion (intrusion on and destruction of adjacent tissues), and sometimes metastasis (spread to other locations in the body via lymph or blood). Examples of cancers include but are not limited to breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, brain cancer, and/or metastasis thereof. The cancer can be any of the above-mentioned types of cancer but especially pancreatic ductal adenocarcinoma, melanoma, prostate cancer, breast cancer, renal cancer, and any combination thereof. When used herein, the term “carcinoma” means cancers derived from epithelial cells. This group includes many of the most common cancers, particularly in the aged, and include nearly all those developing in the breast, prostate, lung, pancreas, and skin.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, diminishment of the reoccurrence of disease, and remission (whether partial or total, including cured), whether detectable or undetectable. “Treating” and “Treatment” can also mean prolonging survival as compared to expected survival if not receiving treatment. “Treating” and “treatment” as used herein also include prophylactic treatment. For example, a subject with early tumor disease can be treated to inhibit growth of a tumor, and thereby prevent progression of tumor growth or alternatively a subject in remission can be treated with a compound or composition described herein to prevent recurrence.

As used herein, the term “subject” refers to a living animal or human in need of treatment for, or susceptible to, a condition involving a tumor disease. The term subject includes, but is not limited to, humans, non-human primates (e.g., chimpanzees, other apes, monkey species), farm animals such as cattle, sheep, pigs, goats and horses, domestic mammals such as dogs and cats, laboratory animals including rodents such as mice, rats and guinea pigs, and the like. The term does not denote a particular age or sex. Thus, adult and newborn subjects, as well as fetuses, whether male or female, are intended to be covered. In preferred embodiments, the subject is a mammal, including humans and non-human mammals. In the most preferred embodiment, the subject is a human.

In some cases, the method comprises administering to a subject a therapeutically effective amount of a compound, pharmaceutically acceptable salt thereof, or a composition comprising one or more compounds described herein. In some cases, the method comprises a single administration, or alternatively comprises a series of applications. For example, in some cases, a compound or composition described herein may be administered at least once a week. However, in another embodiment, the composition may be administered to the subject from about one time per week to about once daily for a given treatment. In another embodiment, the composition is administered twice daily. The length of the treatment period depends on a variety of factors, such as the severity of the disease, the age of the patient, the concentration, the activity of the compounds of the composition described herein, and/or a combination thereof. It will also be appreciated that the effective dosage of the composition used for the treatment or prophylaxis may increase or decrease over the course of a particular treatment or prophylaxis regime. Changes in dosage may result and become apparent by standard diagnostic assays known in the art. In some instances, chronic administration may be required. For example, the composition is administered to the subject in an amount and for a duration sufficient to treat the patient.

A composition comprising a compound (or pharmaceutically acceptable salt thereof) of this disclosure is administered to a subject by any method that achieves the intended purpose or is deemed appropriate by those of skill in the art. For example, a composition of the present invention can be administered as a pharmaceutical, and may be administered systemically or locally via oral or parenteral administration. As used herein, the term “administration” includes oral and parenteral administration. Oral administration includes, for example, administration of oral agents. Such oral agents include, for example, granules, powders, tablets, capsules, solutions, emulsions, and suspensions. Parenteral administration includes, for example, administration of injections. Such injections include, for example, subcutaneous injections, intramuscular injections, and intraperitoneal injection. In some cases, intravenous injections such as drip infusions, intramuscular injections, intraperitoneal injections, subcutaneous injections, suppositories, enemas, oral enteric tablets, or the like can be selected.

Regardless of the route of administration selected, compounds and compositions of this disclosure can be formulated into pharmaceutically acceptable dosage forms by conventional methods known to those of skill in the art.

A therapeutically effective amount relates to the amount of a compound which is sufficient to improve the symptoms, for example a treatment, healing, prevention or improvement of such conditions. In exemplary embodiments, a therapeutically effective amount or dose is an amount such that free antibody is present in the blood. For dosage determinations, it can be advantageous to assess toxicity and therapeutic efficacy of a compound in cell cultures or in experimental animals. For example, the LD₅₀ (i.e., the dose lethal to 50% of the population) and ED₅₀ (i.e., the dose therapeutically effective in 50% of the population) can be determined. From these calculations, dosage ranges for use in humans can be formulated. Dosage ranges can vary depending on factors such as mode of administration.

In some cases, a pharmaceutical composition provided herein comprises one or more compounds of this disclosure, a chemotherapeutic agent or other anti-cancer agent, and a pharmaceutically acceptable carrier. As used herein, the term “chemotherapeutic agent” refers to any substance that, when administered in a therapeutically effective amount to a patient suffering from a tumor or cancer, has a therapeutic beneficial effect on the health and well-being of the patient. A therapeutic beneficial effect on the health and well-being of a patient includes, but it not limited to: (1) curing the cancer; (2) slowing the progress of the cancer; (3) causing the tumor to regress; or (4) alleviating one or more symptoms of the cancer. Chemotherapeutic agents include, without limitation, platinum-based agents, such as cisplatin, gemcitabine, and carboplatin; nitrogen mustard alkylating agents; nitrosourea alkylating agents, such as carmustine (BCNU) and other alkylating agents; antimetabolites, such as methotrexate; purine analog antimetabolites; pyrimidine analog antimetabolites, such as fluorouracil (5-FU) and gemcitabine; hormonal antineoplastics, such as goserelin, leuprolide, and tamoxifen; natural antineoplastics, such as taxanes (e.g., docetaxel and paclitaxel), aldesleukin, interleukin-2, etoposide (VP-16), interferon .alpha., and tretinoin (ATRA); antibiotic natural antineoplastics, such as bleomycin, dactinomycin, daunorubicin, doxorubicin, and mitomycin; and vinca alkaloid natural antineoplastics, such as vinblastine and vincristine.

In some cases, compositions provided herein can additionally comprise one or more other biologically active substances including, without limitation, therapeutic drugs or pro-drugs such as chemotherapeutic agents other than those identified above, scavenger compounds, antibiotics, antiviral agents, antifungal agents, anti-inflammatory agents, vasoconstrictors and anticoagulants, antigens useful for cancer vaccine applications or corresponding pro-drugs.

In interpreting this disclosure, all terms should be interpreted in the broadest possible manner consistent with the context. Unless defined otherwise, all technical and scientific terms used herein have the same meaning as commonly understood by one of ordinary skill in the art to which the invention belongs. It is understood that certain adaptations of the invention described in this disclosure are a matter of routine optimization for those skilled in the art, and can be implemented without departing from the spirit of the invention, or the scope of the appended claims.

So that the compositions and methods provided herein may more readily be understood, certain terms are defined:

As used in this specification and the appended claims, the singular forms “a,” “an,” and “the” include plural references unless the context clearly dictates otherwise. Any reference to “or” herein is intended to encompass “and/or” unless otherwise stated.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The terms “comprising”, “comprises” and “comprised of” as used herein are synonymous with “including”, “includes” or “containing”, “contains”, and are inclusive or open-ended and do not exclude additional, non-recited members, elements, or method steps. The phraseology and terminology used herein is for the purpose of description and should not be regarded as limiting. The use of “including,” “comprising,” “having,” “containing,” “involving,” and variations thereof, is meant to encompass the items listed thereafter and additional items. Embodiments referenced as “comprising” certain elements are also contemplated as “consisting essentially of” and “consisting of” those elements. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

The terms “about” and “approximately” shall generally mean an acceptable degree of error for the quantity measured given the nature or precision of the measurements. Typical, exemplary degrees of error are within 10%, and preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

As used herein, an asterisk “*” or a plus sign “+” may be used to designate the point of attachment for any radical group or substituent group.

The term “alkyl” as contemplated herein includes a straight-chain or branched alkyl radical in all of its isomeric forms, such as a straight or branched group of 1-12, 1-10, or 1-6 carbon atoms, referred to herein as C₁-C₁₂ alkyl, C₁-C₁₀-alkyl, and C₁-C₆-alkyl, respectively.

The term “alkylene” refers to a diradical of an alkyl group. An exemplary alkylene group is —CH₂CH₂—.

The term “haloalkyl” refers to an alkyl group that is substituted with at least one halogen. For example, —CH₂F, —CHF₂, —CF₃, —CH₂CF₃, —CF₂CF₃, and the like.

The term “heteroalkyl” as used herein refers to an “alkyl” group in which at least one carbon atom has been replaced with a heteroatom (e.g., an O, N, or S atom). One type of heteroalkyl group is an “alkoxyl” group.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂-alkenyl, C₂-C₁₀-alkenyl, and C₂-C₆-alkenyl, respectively.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₂-C₁₂-alkynyl, C₂-C₁₀-alkynyl, and C₂-C₆-alkynyl, respectively.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkylene” refers to a diradical of an cycloalkyl group.

The term “partially unsaturated carbocyclyl” refers to a monovalent cyclic hydrocarbon that contains at least one double bond between ring atoms where at least one ring of the carbocyclyl is not aromatic. The partially unsaturated carbocyclyl may be characterized according to the number of ring carbon atoms. For example, the partially unsaturated carbocyclyl may contain 5-14, 5-12, 5-8, or 5-6 ring carbon atoms, and accordingly be referred to as a C₅-C₁₄, C₅-C₁₂, C₅-C₈, or C₅-C₆ membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Exemplary partially unsaturated carbocyclyl groups include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using Cₓ-Cₓ nomenclature where x is an integer specifying the number of ring atoms. For example, a C₃-C₇ heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines, wherein substituents may include, for example, alkyl, cycloalkyl, heterocyclyl, alkenyl, and aryl.

The terms “alkoxyl” or “alkoxy” are art-recognized and refer to an alkyl group, as defined above, having an oxygen radical attached thereto. Representative alkoxyl groups include methoxy, ethoxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and the like.

The term “carbonyl” as used herein refers to the radical —C(O)—.

The term “carboxamido” as used herein refers to the radical —C(O)NRR′, where R and R¹ may be the same or different. Rand R¹ may be independently alkyl, aryl, arylalkyl, cycloalkyl, formyl, haloalkyl, heteroaryl, or heterocyclyl.

The term “carboxy” as used herein refers to the radical —COOH or its corresponding salts, e.g. —COONa, etc.

The term “amide” or “amido” as used herein refers to a radical of the form —R¹C(O)N(R²)—, —R¹C(O)N(R²)R³—, —C(O)NR²R³, or —C(O)NH₂, wherein R¹, R² and R³ are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereo isomers of these compounds and mixtures thereof. Stereoisomers include enantiomers and diastereomers. Mixtures of enantiomers or diastereomers may be designated“(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise. Compositions comprising substantially purified stereoisomers, epimers, or enantiomers, or analogs or derivatives thereof are contemplated herein (e.g., a composition comprising at least about 90%, 95%, or 99% pure stereoisomer, epimer, or enantiomer.)

The invention will be more fully understood upon consideration of the following non-limiting Examples.

### Illustrative Embodiments

The following Embodiments are illustrative and should not be interpreted to limit the scope of the claimed subject matter.

Embodiment 1. A compound of formula (I), or a pharmaceutically acceptable salt thereof:

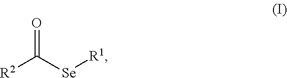

wherein R¹ is selected from a branched or unbranched C₁-C₁₂ alkyl, a branched or unbranched, aryl, or a branched or unbranched C₆-C₁₂ (aryl)alkyl;

wherein R² is selected from a substituted or unsubstituted aryl, a substituted or unsubstituted heteroaryl, a branched or unbranched, substituted or unsubstituted C₆-C₁₂ (heteroaryl)alky or a branched or unbranched, substituted or unsubstituted C₆-C₁₂ (aryl)alkyl, wherein the R² optionally is substituted with one or more R^{A};
wherein R^{A} is selected from halo, —OR^{B}, a branched or unbranched C₁-C₁₂ alkyl, a substituted or unsubstituted C₆-C₁₂ aryl, —NHR^{C}, —C(O)R^{E}, a branched or unbranched, substituted or unsubstituted C₂-C₁₂ (aryl)alkenyl, wherein the aryl and C₂-C₁₂ (aryl)alkenyl are each optionally substituted with R^{F};
   wherein R^{B} is selected from hydrogen, C₁-C₆ alkyl, or C(O)(C₁-C₆ alkyl);
   wherein R^{C} and R^{E} are each independently selected from a branched or unbranched, substituted or unsubstituted C₆-C₁₂ aryl, wherein the C₆-C₁₂ aryl is optionally substituted with R^{H};
   wherein R^{F} and R^{H}, are each independently selected from halo, a branched or unbranched, substituted or unsubstituted C₁-C₁₂ alkyl, or —S(O)(C₁-C₆ alkyl).

Embodiment 2. The compound of claim 1, wherein R¹ is selected from the branched or unbranched C₁-C₁₂ alkyl.

Embodiment 3. The compound of claim 2 or 3, wherein R¹ is selected from the branched or unbranched C₆-C₁₂ arylalkyl, or the aryl.

Embodiment 4. The compound of any of the foregoing claims, wherein R¹ is selected from phenyl or benzyl.

Embodiment 5. The compound of any of the foregoing claims, wherein R² is selected from aryl, the branched or unbranched C₆-C₁₂ (heteroaryl)alky or the branched or unbranched C₆-C₁₂ (aryl)alkyl.

Embodiment 6. The compound of any of the foregoing claims, wherein R² is selected from phenyl, benzyl, naphthalene, -methyl-naphthalene, indol-3-yl, -methyl-1H-indol-3-yl, inden-1-yl, or -1-methyl-1H-indenyl, wherein each R² is substituted with one or more R^{A} groups.

Embodiment 7. The compound of any of the foregoing claims, wherein R^{A} is selected from halo, hydroxyl, —OR^{B}, NHR^{C}, C₁-C₆ alkyl, C(O)R^{E}, phenyl, or benzylidinyl, wherein the phenyl and the benzynylidinyl are each optionally substituted with R^{F}.

Embodiment 8. The compound of any of the foregoing claims, wherein R^{B} is selected from —C(O)C₁-C₆ alkyl or C₁-C₆ alkyl.

Embodiment 9. The compound of any of the foregoing claims, wherein R^{C} and R^{E} are each independently a phenyl substituted with R^{H}.

Embodiment 10. The compound of any of the foregoing claims, wherein R^{F} and R^{H}, are each independently selected from halo, C₁-C₆ alkyl, or S(O)Me.

Embodiment 11. The compound of any of the foregoing claims, of a formula (II):

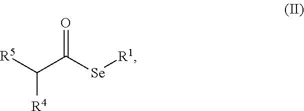

wherein R⁴ is selected from a substituted or unsubstituted aryl or a substituted or unsubstituted C₆-C₁₂ heteroaryl, wherein the C₆-C₁₂ aryl and the C₆-C₁₂ heteroaryl, are each optionally substituted with Rⁱ,
wherein Rⁱ is selected from halo, —OR^{B}, C₁-C₆ alkyl, —C(O)R^{E}, or benzyl, wherein the benzyl is optionally substituted with R^{F},
wherein R^{B} is selected from C₁-C₆ alkyl.
wherein R^{E} is a phenyl substituted with R^{H},
   wherein R^{F} and R^{H}, are each independently selected from halo, C₁-C₆ alkyl, or —S(O)Me;
   wherein R⁵ is selected from hydrogen or C₁-C₆ alkyl.

Embodiment 12. The compound of any of the foregoing claims, wherein the R⁴ branched or unbranched, substituted or unsubstituted C₆-C₁₂ aryl is selected from naphthalene or indolene.

Embodiment 13. The compound of any of the foregoing claims, wherein the R⁴ branched or unbranched, substituted or unsubstituted C₆-C₁₂ heteroaryl is selected from indoline.

Embodiment 14. A compound, or a pharmaceutically acceptable salt thereof, selected from the group consisting of:

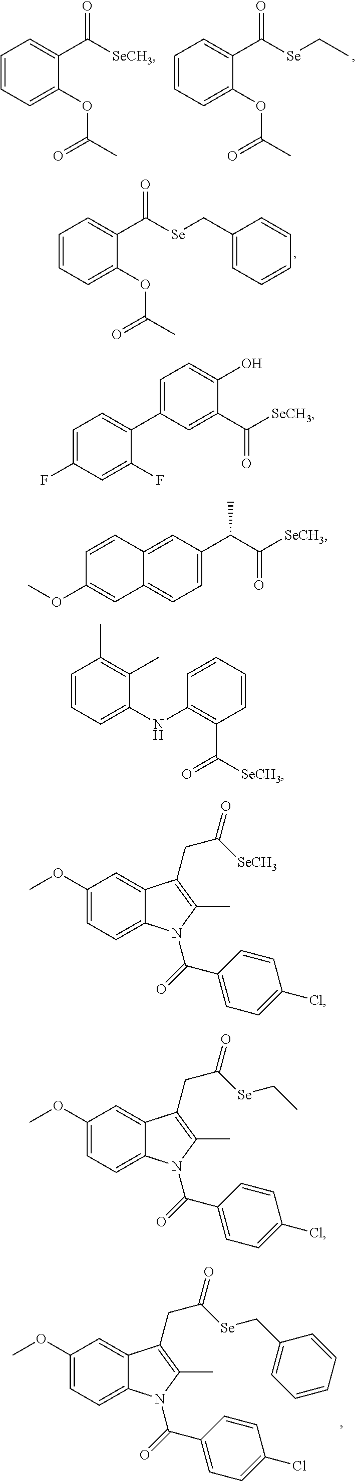

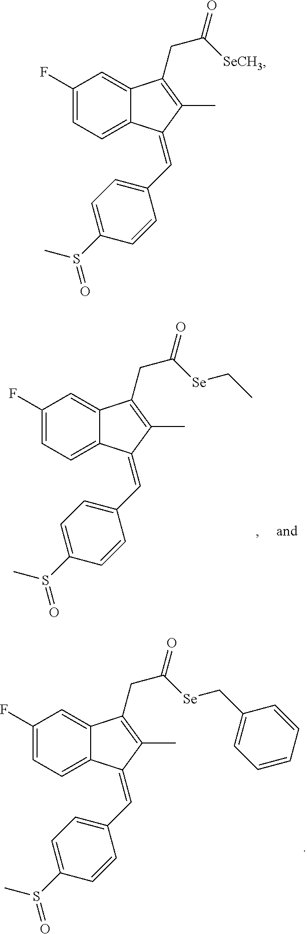

Embodiment 15. A pharmaceutical composition comprising the compound or pharmaceutically acceptable salt of any of the foregoing claims and a pharmaceutically acceptable carrier.

Embodiment 16. A method of treating cancer in a subject having cancer comprising: administering to the subject the compound or pharmaceutically acceptable salt of claim 1 in a therapeutically effective amount to treat the cancer.

Embodiment 17. The method of claim 16, wherein the cancer is selected from the group consisting of pancreatic ductal adenocarcinoma, melanoma, prostate cancer, breast cancer, renal cancer, and any combination thereof.

Embodiment 18. The method of claim 16 or 17, wherein the compound or pharmaceutically acceptable salt is administered in combination with one or more additional anti-cancer therapies.

Embodiment 19. A composition comprising an effective amount of the compound of any of claims 1-14, or pharmaceutically acceptable salts thereof, for use in the inhibition of tumor growth and treatment of cancer.

Embodiment 20. The composition of claim 19, wherein the cancer is selected from the group consisting of pancreatic ductal adenocarcinoma, melanoma, prostate cancer, breast cancer, renal cancer, and any combination thereof.

### EXAMPLES

### Example 1—Design, Synthesis and Biological Evaluation of Novel Se-NSAID Analogs

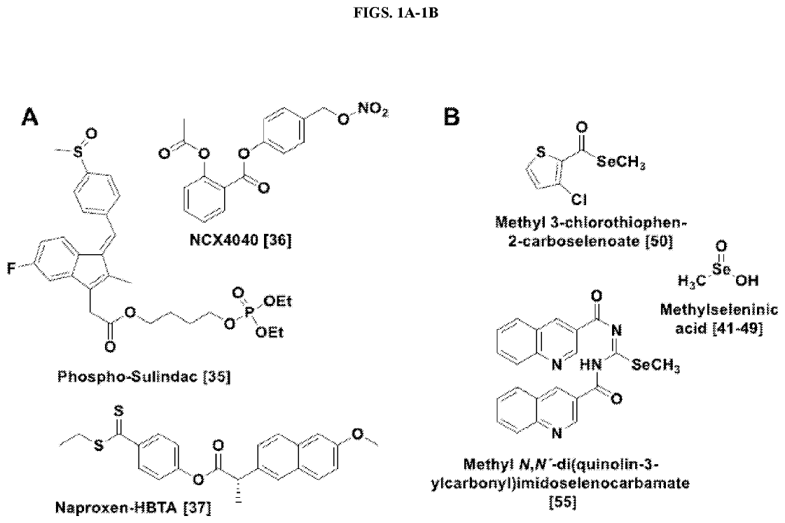

This section describes the design, synthesis and anticancer biological evaluation of novel methylseleno-, ethylseleno- and benzylseleno-nonsteroidal anti-inflammatory drug (NSAID) hybrid molecules. Selenium (Se) is an essential micronutrient for the human health. Among the multiple and complex health benefits ascribed to Se, its role as a cancer prevention agent is the one that has received the greatest attention. Besides, it has been reported that Se supplementation along with anticancer therapies increase the efficacy of standard chemo-therapeutic drugs, as well as limit side effects and improve general condition of patients without reducing effectiveness of the treatment (38, 39). Nevertheless, Se activity is highly dependent on multiple factors, such as dose, metabolic routes and chemical form. Accordingly, elucidating Se metabolism is thus crucial to understand Se activity. Biochemistry of Se encompasses a complex pathway of interrelated intermediates that converge in two main metabolites: methylselenol (CH₃SeH) and hydrogen selenide (H₂Se). CH₃SeH stands out as a key executor for Se anticancer activity, considering that the antitumoral efficacy of a given Se compound might depend on the rate of its metabolic conversion to mono-methylated Se species, presumably CH₃SeH (40). The volatile nature and potent anti-tumor efficacy oblige to the use of CH₃SeH precursors with the ability to release it through cellular metabolism, such as methylseleninic acid (MSA, FIG. 1B). MSA shows potent anti-tumoral activity (41, 42) through inhibition of angiogenesis (43), modulation of MHC class I surface expression (44), induction of apoptosis (45-48) and enhancement of T cell-mediated killing (49), among others. Over the last decade, our research group has reported several methylseleno derivatives with potent anti-tumoral activity, such as methyl 3-chlorothiophen-2-carboselenoate and methyl N,N′-di(quinoline-3-ylcarbonyl)imidoselenocarbamate (FIG. 1B) (50-57). Continuing with this effort and considering the aforementioned chemo-preventive effects of NSAIDs, this example demonstrates that methylseleno precursors derived from NSAIDs (methylseleno-NSAIDs) provide a new, valid, and advantageous approach in the development of promising selective cancer preventive and therapeutic drugs.

As described herein, a new series of 12 seleno-NSAID analogs were developed. Of these, three methylseleno analogs (1a, 5a and 6a) exhibited increase on cytotoxic potency compared with methylselenilic acid and parent NSAID scaffolds (aspirin, indomethacin and sulindac, respectively) alone or in combination in colorectal cancer (CRC) cells. Interestingly, these analogs showed completely different activity profiles towards a National Cancer Institute's (NCI) Developmental Therapeutics Program (DTP) panel of 59 cancer cell lines. Whilst analog 1a only presented negative growth inhibition in one central nervous system (CNS) cell line (RXF-393); analog 5a demonstrated great cytotoxic activity in the most resistant melanoma, prostate, renal and breast cancer cells of the DTP panel. In contrast, 6a possessed low activity towards the 6 most sensitive cells and potent cytotoxicity against the 6 most resistant cells of the DTP panel, with cytotoxic parameter values of 47, 410 and 3900 nM for GI50, TGI and LC50, respectively, in the most resistant cell line (OVCAR-5). Surprisingly, a similarity study using the COMPARE algorithm unveiled an unprecedented behavior for 6a, since this analog only match minimal similarity with romidepsin. Taken together, analog 6a showed unique profile with demonstrated in vitro antitumor efficacy.

Methods

Chemistry. General. Starting materials and solvents were purchased from commercial from commercial suppliers and were used as received. Reaction courses were monitored by thin-layer chromatography (TLC) on precoated silica gel 60 F254 aluminum sheets (Merck, Darmstadt, Germany), and the spots were visualized under UV light. The crude reaction products were purified by silica gel column chromatography using silica gel 60 Å (Merck, 230-400 mesh), and hexane/ethyl acetate was used as the elution solvent. ₁H NMR and ₁₃C NMR spectra were recorded on a Bruker Avance 600 and Bruker Avance Neo 400 instruments in CDCl3. Chemical shifts are reported in δ values (ppm) and coupling constants (J) values are reported in Hz. ₇₇Se NMR spectra were recorded on a Bruker Avance Neo 400 operating at 76 MHz, using Me₂Se as external reference. The signals are quoted as s (singlet), d (doublet), t (triplet), sept (septet), q (quadruplet), m (multiplet), dd (doublet of doublets), and td (triplet of doublets). High-resolution (ESI) MS were carried out at the Chemistry Instrumentation Center, University Park of Pennsylvania at State College, Pa.

General Procedure for the Preparation of Methylseleno, Ethylseleno and Benzylseleno-NSAIDs. To a water solution of sodium hydrogen selenide (2 mmol, 1 equiv), obtained in situ following the procedure described by Sanmartin et al. (68), NSAIDs acyl chloride (2 mmol, 1 equiv) and THF (5 mL) was added. The reaction mixture was stirred at room temperature for 30 min. Then, iodomethane (6.51 mmol, 3 equiv), iodoethane (2 mmol, 1 equiv) or bromobenzene (2 mmol, 1 equiv) were added to the reaction. After 1 h, the reaction mixture was extracted with methylene chloride (3×20 mL). The organic layers were dried with magnesium sulfate and concentrated in vacuo. The crude product was purified using silica gel chromatography with hexane/ethyl acetate gradient (starting from 5% up to 10% ethyl acetate). The acid chloride of Inn, Nap, Dfl, and Mf (2 mmol, 1 equiv) was synthesized by the reaction of the corresponding NSAIDs with oxalyl chloride (6 mmol, 3 equiv) and N,N-Dimethylformamide (0.1 mL) in methylene chloride (20 mL) for 24 h. The acid chloride of sulin was synthesized by the reaction of sulin (2 mmol, 1 equiv) with oxalyl chloride (6 mmol, 3 equiv) in methylene chloride for 3 h.

2-((methylselanyl)carbonyl)phenyl acetate (1a). The title compound was synthesized from solution of sodium hydrogen selenide (2.52 mmol, 1 equiv), O-acetylsalicyloyl chloride (0.5 g, 2.52 mmol) and iodomethane (7.56 mmol, 3 equiv) according to the general procedure described above. A white powder was obtained. Overall yield: 25.9%; mp: 48-49° C. ₁H NMR (600 MHz, CDCl₃) δ: 2.37 (s, 3H, CH₃); 2.38 (s, 3H, Se—CH₃); 7.15 (dd, J=8.1 and 0.8 Hz, 1H); 7.36 (td, J=7.7 and 0.9 Hz, 1H); 7.58 (td, J=7.8 and 1.5 Hz, 1H); 7.94 (dd, J=7.9 and 1.5 Hz, 1H). ₁₃C NMR (100 MHz, CDCl₃) δ: 5.9 (Se—CH₃), 21.2 (CH₃), 124.0, 126.3, 129.7, 131.9, 133.7, 147.2 (aryl), 169.3 (C═O), 192.3 (Se—C═O). ⁷⁷Se NMR (76 MHz, CDCl₃) δ: 487.12. HRMS (ESI) calcd for C₁₀H₁₀O₃Se [M+H]⁺: 258.9795. Found: 258.9863.

2-((ethylselanyl)carbonyl)phenyl acetate (1b). The title compound was synthesized from solution of sodium hydrogen selenide (2.52 mmol, 1 equiv), O-acetylsalicyloyl chloride (0.5 g, 2.52 mmol) and iodoethane (2.52 mmol, 1 equiv) according to the general procedure described above. A yellow oil was obtained. Overall yield: 39.2%. ₁H NMR (400 MHz, CDCl₃) δ: 1.48 (t, J=7.5 Hz, 3H, Se—CH₂—CH₃); 2.35 (s, 3H, CH₃); 3.03 (q, J=7.5 Hz, 2H, Se—CH₂); 7.11 (dd, J=8.1 and 1.1 Hz, 1H); 7.32 (td, J=7.7 and 1.2 Hz, 1H), 7.54 (ddd, J=8.1, 7.5 and 1.6 Hz, 1H); 7.90 (dd, J=7.8 and 1.6 Hz, 1H). ₁₃C NMR (100 MHz, CDCl₃) δ: 15.7 (Se—CH₂—CH₃), 20.2 (Se—CH₂), 21.2 (CH₃), 124.0, 126.3, 129.8, 132.2, 133.6, 147.1 (aryl), 169.3 (C═O), 192.61 (Se—C═O). ₇₇Se NMR (76 MHz, CDCl₃) δ: 598.68. HRMS (ESI) calcd for C₁₁H₁₂O₃Se [M+H]₊: 272.9952. Found: 273.0021.

2-((benzylselanyl)carbonyl)phenyl acetate (1c). The title compound was synthesized from solution of sodium hydrogen selenide (2.52 mmol, 1 equiv), O-acetylsalicyloyl chloride (0.5 g, 2.52 mmol) and bromobenzene (2.52 mmol, 1 equiv) according to the general procedure described above. A white powder was obtained. Overall yield: 58.8%; mp: 59-60° C. ₁H NMR (400 MHz, CDCl₃) δ: 2.31 (s, 3H, CH₃); 4.27 (s, 2H, Se—CH₂); 7.09 (dd, J=8.1 and 1.0 Hz, 1H); 7.21-7.15 (m, 1H), 7.25-7.23 (m, 1H); 7.26-7.25 (m, 1H); 7.28-7.26 (m, 1H); 7.34-7.29 (m, 2H); 7.49 (ddd, J=8.1 and 7.5 and 1.6 Hz, 1H); 7.84 (dd, J=7.9 and 1.6 Hz, 1H). ₁₃C NMR (100 MHz, CDCl₃) δ: 21.3 (CH₃), 29.9 (Se—CH₂), 124.1, 126.4, 127.1, 128.7, 129.1, 129.8, 131.8, 133.9, 138.8, 147.3 (aryl), 169.3 (C═O), 192.1 (Se—C═O). ₇₇Se NMR (76 MHz, CDCl₃) δ: 640.31.

Se-methyl 2′,4′-difluoro-4-hydroxy-[1,1′-biphenyl]-3-carboselenoate (2a). The title compound was synthesized from solution of sodium hydrogen selenide (1.86 mmol, 1 equiv), 2′,4′-difluoro-4-hydroxy-[1,1′-biphenyl]-3-carbonyl chloride (0.5 g, 1.86 mmol) and iodomethane (5.58 mmol, 3 equiv) according to the general procedure described above. A white powder was obtained. Overall yield: 15.6%; mp: 89-90° C. ₁H NMR (400 MHz, CDCl₃) δ: 2.44 (s, 3H, Se—CH₃), 7.03-6.93 (m, 2H); 7.08 (d, J=8.7 Hz, 1H); 7.41 (td, J=8.7 and 6.4 Hz, 1H); 7.64 (dt, J=8.6 and 1.8 Hz, 1H); 7.94 (d, J=0.9 Hz, 1H); 10.81 (s, 1H, OH). ₁₃C NMR (126 MHz, CDCl₃) δ: 5.3 (Se—CH₃), 104.3, 104.5, 104.8, 111.7, 111.9, 118.5, 122.4, 126.6, 130.0, 131.0, 136.4, 157.7 (aryl), 200.6 (Se—C═O). ₇₇Se NMR (76 MHz, CDCl₃) δ: 444.33. HRMS (ESI) calcd for C₁₄H₁₀F₂O₂Se [M+H]₊: 328.9814. Found: 328.9891.

Se-methyl (S)-2-(6-methoxynaphthalen-2-yl)propaneselenoate (3a). The title compound was synthesized from solution of sodium hydrogen selenide (2.01 mmol, 1 equiv), 2-(6-methoxynaphthalen-2-yl)propanoyl chloride (0.5 g, 2.01 mmol) and iodomethane (6.03 mmol, 3 equiv) according to the general procedure described above. A white powder was obtained. Overall yield: 28.39%; mp: 60-6° C. ₁H NMR (400 MHz, CDCl₃) δ: 1.65 (d, J=7.1 Hz, 3H, CH₃); 2.18 (s, J=5.4 Hz, 3H, Se—CH₃); 3.95 (d, J=3.5 Hz, 3H, O—CH₃); 4.06 (q, J=7.1 Hz, 1H, CH); 7.15 (d, J=2.4 Hz, 1H); 7.18 (dd, J=8.9 and 2.5 Hz, 1H); 7.41 (dd, J=8.5 and 1.9 Hz, 1H); 7.73 (d, J=1.1 Hz, 1H); 7.74 (d, J=3.2 Hz, 1H); 7.76 (d, J=3.6 Hz, 1H). ₁₃C NMR (100 MHz, CDCl₃) δ: 5.3 (Se—CH₃), 18.0 (CH₃), 55.3 (O—CH₃), 57.7 (CH), 105.7, 119.1, 126.7, 127.2, 127.3, 128.9, 129.4, 134.1, 134.3, 157.9 (aryl), 204.42 (Se—C═O). ₇₇Se NMR (76 MHz, CDCl₃) δ: 460.09. HRMS (ESI) calcd for C₁₅H₁₆O₂Se [M+H]₊: 309.0316. Found: 309.0395.

Se-methyl 2-((2,3-dimethylphenyl)amino)benzoselenoate (4a). The title compound was synthesized from solution of sodium hydrogen selenide (1.93 mmol, 1 equiv), 2-((2,3-dimethylphenyl)amino)benzoyl chloride (0.5 g, 1.93 mmol) and iodomethane (5.79 mmol, 3 equiv) according to the general procedure described above. A yellow powder was obtained. Overall yield: 3.4%; mp: 83-85° C. ₁H NMR (600 MHz, CDCl₃) δ: 2.18 (s, 3H, CH₃); 2.35 (s, 3H, CH₃); 2.39 (s, 3H, Se—CH₃); 6.73 (t, J=7.8 Hz, 2H); 7.06 (d, J=7.1 Hz, 1H); 7.14 (dd, J=16.2 and 11.1 Hz, 2H); 7.27 (d, J=7.7 Hz, 1H); 7.92 (d, J=7.6 Hz, 1H); 9.34 (s, 1H, NH). ₁₃C NMR (151 MHz, CDCl₃) δ: 5.2 (Se—CH₃), 14.0 (CH₃), 20.6 (CH₃), 114.2, 116.5, 120.5, 123.4, 126.0, 127.2, 131.6, 132.7, 134.6, 138.2, 138.3, 146.2 (aryl), 196.1 (Se—C═O). ₇₇Se NMR (76 MHz, CDCl₃) δ: 439.64. HRMS (ESI) calcd for C₁₆H₁₇NOSe [M+H]₊: 320.0475. Found: 320.0555.

Se-methyl 2-(1-(4-chlorobenzoyl)-5-methoxy-2-methyl-1H-indol-3-yl)ethane-selenoate (5a). The title compound was synthesized from solution of sodium hydrogen selenide (2.66 mmol, 1 equiv), 2-(1-(4-chlorobenzoyl)-5-methoxy-2-methyl-1H-indol-3-yl)acetyl chloride (1 g, 2.66 mmol) and iodomethane (7.97 mmol, 3 equiv) according to the general procedure described above. A white powder was obtained. Overall yield: 43.3%; mp: 90-91° C. ₁H NMR (400 MHz, CDCl₃) δ: 2.17 (s, 3H, Se—CH₃); 2.40 (s, 3H, CH₃); 3.83 (s, 3H, O—CH₃); 3.86 (s, 2H, CH₂); 6.70 (dd, J=9.1 and 2.4 Hz, 1H); 6.91 (d, J=2.6 Hz, 1H); 6.92 (s, 1H); 7.48 (d, J=8.3 Hz, 2H); 7.67 (d, J=8.3 Hz, 2H). ₁₃C NMR (100 MHz, CDCl₃) δ: 5.2 (Se—CH₃), 13.5 (CH₃), 42.6 (CH₂), 55.7 (O—CH₃), 101.1, 11.6, 112.0, 115.1, 129.2, 130.2, 130.9, 131.3, 133.7, 137.2, 139.5, 156.2 (aryl), 168.3 (C═O), 200.5 (Se—C═O). ₇₇Se NMR (76 MHz, CDCl₃) δ: 479.73. HRMS (ESI) calcd for C₂₀H₁₈ClNO₃Se [M+H]₊: 436.0140. Found: 436.0207.

Se-ethyl 2-(1-(4-chlorobenzoyl)-5-methoxy-2-methyl-1H-indol-3-yl)ethane-selenoate (5b). The title compound was synthesized from solution of sodium hydrogen selenide (2.66 mmol, 1 equiv), 2-(1-(4-chlorobenzoyl)-5-methoxy-2-methyl-1H-indol-3-yl)acetyl chloride (1 g, 2.66 mmol) and iodoethane (2.66 mmol, 1 equiv) according to the general procedure described above. A white powder was obtained. Overall yield: 34.0%; mp: 98-99° C. ₁H NMR (400 MHz, CDCl₃) δ: 1.37 (t, J=7.5 Hz, 3H, Se—CH₂—CH₃); 2.39 (s, 3H, CH₃); 2.86 (q, J=7.5 Hz, 2H, Se—CH₂), 3.84 (s, 3H, O—CH₃); 3.86 (s, 2H, CH₂); 6.69 (dd, J=9.0 and 2.4 Hz, 1H); 6.90 (s, 1H); 6.92 (d, J=2.8 Hz, 1H), 7.48 (d, J=8.4 Hz, 2H); 7.67 (d, J=8.4 Hz, 2H). _{13C NMR (151 MHz, CDCl3)} δ: 13.6 (CH₃), 15.6 (Se—CH₂—CH₃), 19.6 (Se—CH₂), 43.1 (CH₂), 55.7 (O—CH₃), 101.1, 111.8, 111.9, 115.0, 129.2, 130.63, 130.9, 131.3, 133.8, 137.1, 139.4, 156.2 (aryl), 168.3 (C═O), 200.5 (Se—C═O). ₇₇Se NMR (76 MHz, CDCl₃) δ: 591.71. HRMS (ESI) calcd for C₂₁H₂₀ClNO₃Se [M+H]₊: 450.0297. Found: 450.0370.

Se-benzyl 2-(1-(4-chlorobenzoyl)-5-methoxy-2-methyl-1H-indol-3-yl)ethane-selenoate (5c). The title compound was synthesized from solution of sodium hydrogen selenide (2.66 mmol, 1 equiv), 2-(1-(4-chlorobenzoyl)-5-methoxy-2-methyl-1H-indol-3-yl)acetyl chloride (1 g, 2.66 mmol) and bromobenzene (2.66 mmol, 1 equiv) according to the general procedure described above. A white powder was obtained. Overall yield: 22.5%; mp: 101-102° C. ₁H NMR (400 MHz, CDCl₃) δ: 2.37 (s, 3H, CH₃); 3.82 (s, 3H, O—CH₃); 3.88 (s, 2H, CH₂); 4.11 (s, 2H, Se—CH₂); 6.68 (dd, J=9.1 and 2.4 Hz, 1H); 6.89 (dd, J=5.7 and 3.2 Hz, 2H); 7.34-7.15 (m, 6H); 7.46 (d, J=8.5 Hz, 2H); 7.65 (d, J=8.5 Hz, 2H). ₁₃C NMR (100 MHz, CDCl₃) δ: 13.6 (CH₃), 29.2 (Se—CH₂); 42.7 (CH₂); 55.7 (O—CH₃), 101.0, 111.4, 112.1, 115.0, 127.0, 128.5, 128.6, 128.9, 129.0, 129.0, 129.1, 130.6, 131.3, 133.7, 137.3, 138.8, 156.2 (aryl), 168.3 (C═O), 200.2 (Se—C═O). ₇₇Se NMR (76 MHz, CDCl₃) δ: 636.21. HRMS (ESI) calcd for C₂₆H₂₂ClNO₃Se [M+H]₊: 512.0453. Found: 512.0521.

Se-methyl (Z)-2-(5-fluoro-2-methyl-1-(4-(methylsulfinyl)benzylidene)-1H-inden yl)ethaneselenoate (6a). The title compound was synthesized from solution of sodium hydrogen selenide (2.65 mmol, 1 equiv), (Z)-2-(5-fluoro-2-methyl-1-(4-(methylsulfinyl)benzylidene)-2,7a-dihydro-1H-inden-3-yl)acetyl chloride (1 g, 2.65 mmol) and iodomethane (7.96 mmol, 3 equiv) according to the general procedure described above. A yellow powder was obtained. Overall yield: 8.5%; mp: 77-78° C. ₁H NMR (600 MHz, CDCl₃) δ: 2.18 (s, 3H, Se—CH₃); 2.23 (s, 3H, CH₃); 2.54 (s, 3H, O═S—CH₃); 3.79 (s, 2H, CH₂); 6.59 (td, J=8.9 and 2.4 Hz, 1H), 6.85 (dd, J=8.9 and 2.4 Hz, 1H), 7.18 (s, 1H), 7.29 (d, J=8.3 Hz, 2H), 7.38 (dd, J=8.4 and 5.1 Hz, 1H), 7.45 (d, J=8.2 Hz, 2H). _{13C NMR (151 MHz, CDCl3)} δ: 5.1 (Se—CH₃), 10.9 (CH₃), 15.4 (O═S—CH₃), 44.4 (CH₂), 105.7, 105.9, 110.8, 110.9, 123.8, 123.8, 126.0, 130.0, 130.6, 132.9, 139.4, 140.0, 140.1, 146.3, 162.3, 164.0 (aryl), 199.0 (Se—C═O). ₇₇Se NMR (76 MHz, CDCl₃) δ: 479.82.

Se-ethyl (Z)-2-(5-fluoro-2-methyl-1-(4-(methylsulfinyl)benzylidene)-1H-inden-3-yl)ethaneselenoate (6b). The title compound was synthesized from solution of sodium hydrogen selenide (2.65 mmol, 1 equiv), (Z)-2-(5-fluoro-2-methyl-1-(4-(methylsulfinyl)benzylidene)-2,7a-dihydro-1H-inden-3-yl)acetyl chloride (1 g, 2.65 mmol) and iodoethane (2.65 mmol, 1 equiv) according to the general procedure described above. A yellow powder was obtained. Overall yield: 14.2%; mp: 60-61° C. ₁H NMR (400 MHz, CDCl₃) δ: 1.38 (t, J=7.5 Hz, 3H, Se—CH₂—CH₃); 2.23 (s, 3H, CH₃); 2.55 (s, 3H, O═S—CH₃); 2.87 (q, J=7.5 Hz, 2H, Se—CH₂); 3.78 (s, 2H, CH₂); 6.59 (ddd, J=9.1, 8.6 and 2.4 Hz, 1H); 6.86 (dd, J=8.9 and 2.4 Hz, 1H); 7.18 (s, 1H); 7.29-7.27 (m, 1H); 7.31-7.29 (m, 1H); 7.39 (dd, J=8.4 and 5.2 Hz, 1H); 7.46 (d, J=8.0 Hz, 2H). ₁₃C NMR (100 MHz, CDCl₃) δ: 10.88 (CH₃), 15.40, 15.62 (O═S—CH₃, Se—CH₂—CH₃), 19.59 (Se—CH₂), 44.73 (CH₂), 105.7, 105.9, 110.7, 110.9, 123.7, 123.8, 126.0, 130.0, 130.5, 130.5, 132.9, 139.3, 140.0, 146.37, 161.89, 164.34 (aryl), 199.1 (Se—C═O). ₇₇Se NMR (76 MHz, CDCl₃) δ: 592.11.

Se-benzyl (Z)-2-(5-fluoro-2-methyl-1-(4-(methylsulfinyl)benzylidene)-1H-inden-3-yl)ethaneselenoate (6c). The title compound was synthesized from solution of sodium hydrogen selenide (2.65 mmol, 1 equiv), (Z)-2-(5-fluoro-2-methyl-1-(4-(methylsulfinyl)benzylidene)-2,7a-dihydro-1H-inden-3-yl)acetyl chloride (1 g, 2.65 mmol) and bromobenzene (2.65 mmol, 1 equiv) according to the general procedure described above. A yellow powder was obtained. Overall yield: 12.8%; mp: 87-88° C. ₁H NMR (400 MHz, CDCl₃) δ: 2.21 (s, 3H, CH₃); 2.54 (s, 3H, O═S—CH₃); 3.80 (s, 2H, CH₂); 4.12 (s, 2H, Se—CH₂); 6.59 (td, J=8.9 and 2.3 Hz, 1H), 6.84 (dd, J=8.9, 2.4 Hz, 1H), 7.32-7.15 (m, 8H), 7.38 (dd, J=8.4, 5.2 Hz, 1H), 7.45 (d, J=8.1 Hz, 2H). ₁₃C NMR (100 MHz, CDCl₃) δ: 10.9 (CH₃), 15.4 (O═S—CH₃), 29.2 (Se—CH₂), 44.3 (CH₂), 105.7, 105.9, 110.7, 111.0, 123.8, 123.9, 125.9, 127.0, 128.6, 128.9, 129.7, 129.7, 129.9, 130.7, 132.8, 138.7, 139.4, 140.0, 140.3, 146.2, 161.9, 164.3 (aryl), 198.8 (Se—C═O). ₇₇Se NMR (76 MHz, CDCl₃) δ: 636.03.

Cell Culture Conditions. The cell lines were obtained from the American Type Culture Collection (ATCC). PANC-1 and Caco-2 cell lines were maintained in DMEM medium (Gibco); HT-29, HCT-116 and RKO cells were maintained in McCoy's 5A medium (Gibco); and DU-145 and H1299 cells were maintained in RPMI 1640 medium (Gibco), supplemented with 10% fetal bovine serum (FBS; Gibco) and 1% antibiotics (10.00 units/mL penicillin and 10.00 mg/mL streptomycin; Gibco). Cells were preserved in tissue culture flasks at 37° C. and 5% CO₂. Culture medium was replaced every three days.

Cell Viability Assay. The effect of each compound on cell viability was tested using the MTT assay (81). Briefly, 9000 cells/well were grown in 96-well plates for 12 h. Then these cells were incubated with either DMSO (control) or nine different concentration between 0.01-50 μM of the test compounds for 24, 48, and 72 h. Three hours before the termination point, 20 μL of MTT were added to measure cellular viability. The resultant formazan crystals were dissolved in 50 μL of DMSO, and absorbance was measured at 570 nm and 630 nm wavelengths. IC50, GI50, TGI and LC50 values were calculated using OriginPro 8.5.1.

Cell Cycle Analysis. Cell cycle analyses were determined by following a flow cytometry protocol as described earlier (82). In brief, HT-29 cells were serum deprived for 72 hours to get them synchronized. At the end of 72 hours, cells were supplied with fresh medium or fresh medium with 1, 2.5, 5 and 10 μM of 1a and 6a for 24 hours. After each time point, the cells were fixed in ice cold 70% ethanol. Fixed cells were washed with 1×PBS and suspended in PI staining solution. PI staining solution was made by adding 0.1% (v/v) triton X-100, 20 mg of DNase-free RNase A, and 2 mg of PI in 100 mL of PBS. After staining for 15 min, the cells were analyzed by flow cytometer (Coulter Epics XL, Beckman Coulter).

Protein Expression Analysis. Western Blot was performed to assess the expression level of different proteins before and after treatment. In brief, HT-29 cells were treated with the compound for different time intervals and the whole-cell lysates were made by lysing cells using RIPA buffer (Thermo Scientific, USA). Protease (Roche, USA) and phosphatase inhibitor cocktails (Sigma-Aldrich, USA) were used to prevent the degradation of the proteins present in the lysate. The resulting lysates were incubated on ice for 30 min and spun at 10,000 rpm for 15 min to pellet down cell debris. The total protein concentration was determined using Bradford assay (Thermo Scientific, USA). Equal amounts of cell lysates were resolved using NuPAGE gel 4-12% (Life Technologies, Carlsbad, Calif.). The resolved proteins were electro-transferred to a PVDF membrane and different antibodies were used to probe the protein expression levels. Enhanced Chemiluminescent reagent (Life technologies, USA) was used to detect the protein of interest.

Apoptosis assay. Induction of apoptosis after the treatment with compounds 1a and 6a were analyzed using Annexin V & Dead cell assay kit and Caspase 3/7 Assay kit (EMD Millipore, Billercia, Mass.) using Muse cell analyzer following manufacturer's protocol. Briefly, the HT-29 cells were plated in triplicates in a six-well plate at densities of 1×10⁵ cells/well. Following day, the cells were treated with the compounds 1a and 6a and incubated for 48 h. At the end of the treatment duration, the cells were prepared following manufacturer's guidelines and the data were acquired using Muse 1.4 software.

NCI-60 Analysis. 1a and 6a were submitted to National Cancer Institute's (NCI) Developmental Therapeutics Program (DTP) where the cytotoxicity profile was investigated across their 60 human cancer cell line panel according to the protocol available on multiple sources (71-73, 83). The COMPARE algorithm (73), was utilized to evaluate the activity profile of 6a in comparison to those of known cytotoxins established in marketed drugs and standard agent data sets in order to correlate plausible modes of action.

Results and Discussion

Design. Several NSAIDs, such as ASA, Sulin and Naproxen (Nap), have been shown to be effective chemo-preventive drugs in both pre-clinical and clinical studies on various cancers, including CRC (2, 5, 7, 19-21, 58). In addition, several Se derivatives, covering methylseleno analogs, demonstrated chemo-preventive and chemo-therapeutic activities in different preclinical models (59). More recently, Se showed synergistic effect with Sulin to inhibit intestinal tumorigenesis (60). Moreover, the addition of Se moieties into NSAID structures (Se-NSAIDs) yielded novel analogs with an outstanding increase in the anti-tumor effect with respect to parent NSAIDs, together with great selectivity toward cancer cells (61, 62). Based on all these facts, we hypothesized that assembling a potential precursor molecule gathering both CH₃SeH and NSAIDs could be a valid approach in the development of selective cancer preventive and therapeutic drugs. Therefore on a first approach a total of 6, novel molecules were designed by incorporating a Se-methyl group into the framework of several NSAIDs (FIG. 2) using a selenoester bond (50, 63). This linking bond was chosen since it might be easily cleaved inside the cell, given its analogy with ester bond (64). Thus, six different NSAIDs were selected for methylselenoester incorporation with the purpose of first identifying the NSAID that formed the most effective anticancer hybrid molecules (FIG. 2). The selected NSAIDs encompass a variety of chemical structures, such as salicylates (ASA and diflunisal, Dfl), arylpropionic (Nap), phenamates (mefenamic acid, Mf) and acetates (Inn and Sulin). These chemical differences could lead to different pharmacokinetic parameters (65-67).

The in vitro cytotoxicity results (Table 1) showed a three-fold increase on cytotoxic potency of 1a, 5a and 6a compared with parent NSAID scaffolds (ASA, Inn and Sulin, respectively) in CRC cells (HT-29). To demonstrate whether this effect solely was caused by the methylated form of Se or unrestricted to other alkyl or aromatic forms of Se, a series 2 of analogs, e.g., the ethylseleno and benzylseleno derivatives of 1a, 5a and 6a, were also designed (FIG. 2).

Chemistry. Methylseleno-, ethylseleno- and benzylseleno-analogs of ASA (1), Dfl (2), Nap (3), Mf (4), Inn (5) and Sulin (6) were obtained following the reported methodology with minor modifications (68, 69). The synthetic procedures for the preparation of these derivatives are summarized in Scheme 1. First, the corresponding acid chloride of NSAIDs in THF was reacted with an aqueous solution of sodium hydrogen selenide (NaHSe). The resulting sodium salts of the corresponding NSAID were then reacted in situ with 3 eq. of iodomethane or 1 eq. of iodoethane or benzyl chloride. These novel derivatives were purified by silica gel column chromatography using a mixture of n-hexane and ethyl acetate as eluent. Compounds 4 and 6 presented troublesome synthesis with poor yields (<14.19%). These poor yields could be due to decomposition in the formation of the NSAID-acid chloride.

The structures of all the compounds were confirmed using spectroscopic methods (₁H NMR, ₁₃C NMR) and high-resolution mass spectrometry (HRMS). Regarding ¹H NMR, two microsatellites reflecting the coupling of ₇₇Se with ₁H (₂J_{Se-H}=64.1-73.3; ₃J_{Se-H}=5.2-5.6 Hz) were characteristics of the methylseleno group, whose chemical shift appeared between 2.17 and 2.44 ppm. Concerning ₁₃C NMR, chemical shift of methylseleno, ethylseleno or benzylseleno groups appeared at the following ranges 5.1-5.9, 19.6-20.2 or 29.2-29.9 ppm, respectively. Inspection of ₇₇Se NMR spectra revealed one sharp peak in the ranges of 479.73-487.12, 591.71-598.68 or 636.03-640.31 ppm, for the methylseleno, ethylseleno or benzylseleno groups, respectively.

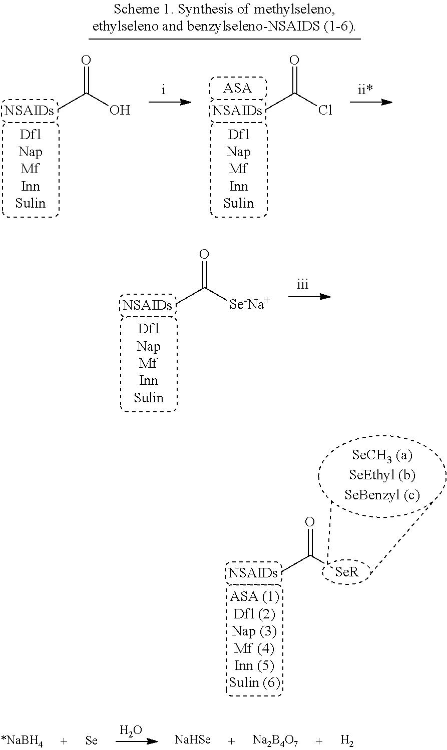

Evaluation of the antiproliferative activities of the novel molecules. The anticancer activity of six methylseleno-NSAIDs (1a-6a) and the corresponding parent NSAIDs (ASA, Dfl, Nap, Mf, Inn and Sulin) were examined in vitro on several human tumor cell lines: PANC-1 (pancreatic carcinoma), HT-29 (CRC), DU-145 (prostate carcinoma) and H1299 (lung carcinoma) using the 3-(4,5-dimethylthiazol-2-yl)-2,5-diphenyltetrazolium bromide (MTT) assay (70). These four cell lines were treated with each compound at three concentrations (5, 25 and 50 μM) for 48 h. As presented in Table 1, parent NSAIDs had no effect on cancer cell viability up to the maximum tested dose. Five methylseleno-NSAIDs analogs (1a, 2a, 4a, 5a, and 6a) demonstrated reduction of the cell growth by >70% at 25 μM in at least one cell line. CRC cells (HT-29) were shown to be the most sensitive cells to treatments.

The inhibition of cell viability on HT-29 obtained for the methylseleno-NSAIDs analogs 1a-6a, compared to their corresponding parent NSAIDs, showed that the introduction of the methylseleno moiety in ASA (1a), Inn (5a) and Sulin (6a) resulted in significantly more potent analogs with ˜75-80% inhibition in cell viability at 5 μM. On the contrary, similar modification over Dfl, Nap and Mf (2a, 3a, and 4a) were relatively less effective on CRC viability at this dose (Table 1).

TABLE 1
Anti-proliferative effect of methylseleno-NSAIDs
analogs (1a-6a) compared to their respective parent NSAIDs
on cancer cell viability after 48 hours of treatment.
Cell line

| | Dose | | | | |
|---|---|---|---|---|---|
| Comp. | (μM) | Panc-1 | HT-29 | DU145 | H1299 |
| 1a | 5 | 43.7 ± 12.1 | 20.8 ± 5.6 | 43.2 ± 1.8 | 48.7 ± 5.0 |
| | 25 | 36.9 ± 6.3 | 18.5 ± 4.1 | 35.9 ± 7.0 | 41.6 ± 7.2 |
| | 50 | 36.8 ± 6.8 | 17.7 ± 12.6 | 36.5 ± 6.7 | 31.4 ± 7.7 |
| 2a | 5 | 53.5 ± 9.7 | 46.6 ± 11.9 | 53.9 ± 14.1 | 96.8 ± 19.8 |
| | 25 | 38.5 ± 8.1 | 23.5 ± 7.2 | 40.7 ± 8.3 | 80.0 ± 10.7 |
| | 50 | 39.1 ± 11.1 | 28.4 ± 2.7 | 39.5 ± 2.7 | 79.9 ± 9.0 |
| 3a | 5 | 46.7 ± 9.7 | 52.2 ± 30.9 | 56.2 ± 18.4 | 48.3 ± 5.4 |
| | 25 | 34.4 ± 6.3 | 41.8 ± 26.4 | 35.0 ± 16.1 | 42.4 ± 2.7 |
| | 50 | 30.5 ± 6.0 | 46.4 ± 24.7 | 31.4 ± 23.6 | 34.3 ± 1.3 |
| 4a | 5 | 47.5 ± 5.9 | 41.6 ± 6.4 | 58.0 ± 14.1 | 98.7 ± 0.4 |
| | 25 | 26.3 ± 3.9 | 16.6 ± 4.7 | 29.7 ± 10.9 | 99.4 ± 0.3 |
| | 50 | 22.6 ± 3.0 | 22.6 ± 0.7 | 32.0 ± 6.5 | 100 ± 22.4 |
| 5a | 5 | 61.9 ± 4.4 | 26.3 ± 2.9 | 52.1 ± 1.5 | 49.5 ± 9.4 |
| | 25 | 66.6 ± 1.9 | 17.9 ± 3.7 | 34.5 ± 3.0 | 37.0 ± 5.6 |
| | 50 | 60.2 ± 5.5 | 18.0 ± 3.5 | 27.4 ± 1.7 | 32.4 ± 5.8 |
| 6a | 5 | 49.4 ± 20.1 | 22.9 ± 2.9 | 60.1 ± 2.2 | 53.4 ± 20.2 |
| | 25 | 29.0 ± 9.5 | 11.9 ± 6.6 | 44.9 ± 2.4 | 41.9 ± 14.9 |
| | 50 | 21.3 ± 9.2 | 11.1 ± 6.2 | 52.5 ± 2.9 | 31.3 ± 6.4 |
| ASA | 5 | 86.7 ± 5.4 | 73.1 ± 13.0 | 89.8 ± 11.0 | 44.1 ± 4.4 |
| | 25 | 63.8 ± 3.1 | 57.2 ± 11.3 | 85.3 ± 25.1 | 51.1 ± 1.6 |
| | 50 | 68.6 ± 5.1 | 35.5 ± 7.7 | 90.9 ± 24.6 | 58.3 ± 3.6 |
| Dfl | 5 | 77.9 ± 5.4 | 93.2 ± 5.7 | 71.0 ± 6.9 | 85.2 ± 3.6 |
| | 25 | 75.5 ± 6.9 | 82.7 ± 9.8 | 71.5 ± 9.1 | 94.3 ± 3.9 |
| | 50 | 68.5 ± 9.3 | 74.7 ± 2.0 | 78.9 ± 6.4 | 88.7 ± 7.8 |
| Nap | 5 | 89.6 ± 1.2 | 97.2 ± 3.9 | 100 ± 8.4 | 100 ± 4.9 |
| | 25 | 91.1 ± 9.8 | 99.6 ± 5.5 | 100 ± 10.0 | 100 ± 12.6 |
| | 50 | 95.7 ± 26.0 | 98.4 ± 3.2 | 100 ± 9.5 | 100 ± 23.1 |
| Mf | 5 | 72.4 ± 5.6 | 91.9 ± 4.9 | 100 ± 20.5 | 71.4 ± 15.5 |
| | 25 | 61.9 ± 10.9 | 75.7 ± 4.1 | 100 ± 31.1 | 44.9 ± 1.4 |
| | 50 | 65.9 ± 5.1 | 64.9 ± 4.5 | 100 ± 37.9 | 39.4 ± 1.3 |
| Inn | 5 | 94.1 ± 8.3 | 100 ± 8.4 | 89.6 ± 12.3 | 76.0 ± 23.7 |
| | 25 | 85.9 ± 10.5 | 99.8 ± 14.2 | 97.6 ± 21.0 | 70.4 ± 19.8 |
| | 50 | 83.2 ± 6.3 | 98.3 ± 13.4 | 96.2 ± 18.5 | 66.2 ± 14.0 |
| Sulin | 5 | 88.0 ± 22.5 | 99.1 ± 2.4 | 82.2 ± 1.9 | 87.5 ± 2.5 |
| | 25 | 90.6 ± 28.9 | 97.3 ± 2.1 | 83.2 ± 7.2 | 72.5 ± 2.7 |
| | 50 | 84.5 ± 32.7 | 98.6 ± 3.1 | 85.5 ± 3.7 | 84.9 ± 12.1 |

Data represent the mean ± SD values for cell viability expressed as %, determined by the MTT assay in triplicates.

As shown in Table 1, HT-29 was the most sensitive cell line to these compounds. Furthermore, compounds 1a, 5a, and 6a were found to be the most active in HT-29, with reduction of the cell growth by >70% after 48 h of treatment at 5 μM. They were further tested at nine different concentrations (0.01, 0.1, 0.5, 1, 2.5, 5, 10, 10, 25 and 50 μM), in order to establish their dose-response curves against a panel of four different human CRC cells (HT-29, HCT-116, RKO and CACO-2) at 24, 48 and 72 h of treatment (Table 2). Our data showed that these derivatives effectively inhibited cell viability of mostly all the CRC cells tested as compared to the parent NSAID, with IC₅₀ values ranging from 0.7 to 19 μM at 48 and 72 h (Table 2). HT-29 and CACO-2 cells demonstrated to be more sensitive towards these analogs compared to RKO and HTC-116. Furthermore, 1a and 6a analogs exhibited outstanding cytotoxic activity over HT-29 and CACO-2 cells, with IC₅₀ values in the low micromolar range. In addition, 1a did not exhibit cytotoxicity towards RKO cells even at the highest concentration (50 μM) tested up to 72 h (Table 2).

TABLE 2
IC₅₀ values (μM) for 1a, 5a and 6a analogs and
their respective parent NSAIDs in different CRC cells.
Cell line

| | Time | | | | |
|---|---|---|---|---|---|
| Comp. | (h) | HT-29 | HCT-116 | RKO | CACO-2 |
| 1a | 24 | 1.9 ± 0.6 | >50 | >50 | 2.6 ± 0.2 |
| | 48 | 1.2 ± 0.1 | 2.2 ± 0.3 | >50 | 0.9 ± 0.1 |
| | 72 | 1.2 ± 0.4 | 1.0 ± 0.2 | >50 | 0.8 ± 0.1 |
| 5a | 24 | >50 | >50 | >50 | >50 |
| | 48 | 3.3 ± 0.9 | 5.6 ± 2.6 | 10.1 ± 6.9 | 4.2 ± 1.7 |
| | 72 | 3.3 ± 0.3 | 18.7 ± 4.9 | 11.6 ± 5.9 | 5.5 ± 1.1 |
| 6a | 24 | 2.4 ± 0.3 | 7.3 ± 2.3 | >50 | 3.0 ± 0.5 |
| | 48 | 2.0 ± 0.7 | 3.2 ± 0.7 | 17.2 ± 2.4 | 1.3 ± 0.6 |
| | 72 | 1.4 ± 0.3 | 1.8 ± 0.6 | 17.3 ± 0.8 | 0.7 ± 0.2 |
| ASA | 24 | >50 | >50 | >50 | >50 |
| | 48 | >50 | >50 | >50 | >50 |
| | 72 | >50 | >50 | >50 | >50 |
| Inn | 24 | >50 | >50 | >50 | >50 |
| | 48 | >50 | >50 | >50 | >50 |
| | 72 | >50 | >50 | >50 | >50 |
| Sulin | 24 | >50 | >50 | >50 | >50 |
| | 48 | >50 | >50 | >50 | >50 |
| | 72 | >50 | >50 | >50 | >50 |

Data represent the mean IC₅₀ (half maximal inhibitory concentration) ± SD values for cell viability expressed as %, determined by the MTT assay in triplicates.

Based on the cytotoxicity results, three parameters (GI₅₀, TGI, LC₅₀) were further calculated for these three analogs and their parent NSAIDs against HT-29 cell line (Table 3). Interestingly, 1a and 6a analogs displayed impressive cytotoxic potential over HT-29, with GI₅₀, TGI, LC₅₀ values in the low μM range. Remarkably, 1a exhibited this cytotoxic potential even at a short period of time (24 h). However, 5a analog inhibited the cell growth, without leading to cell death (Table 3).

TABLE 3
GI₅₀, TGI and LC₅₀ values (μM) of 1a, 5a and 6a analogs
and their respective parent NSAIDs in HT-29 cancer cell line.

| | Time | HT-29 | | |
|---|---|---|---|---|
| Comp. | (h) | GI50 (μM) | TGI (μM) | LC50 (μM) |
| 1a | 24 h | 0.1 ± 0.1 | 0.5 ± 0.3 | 1.7 ± 1.6 |
| | 48 h | 0.1 ± 0.1 | 0.2 ± 0.1 | 0.7 ± 0.1 |
| | 72 h | 0.2 ± 0.1 | 0.7 ± 0.3 | 2.5 ± 1.4 |
| 5a | 24 h | 4.9 ± 2.2 | >50 | >50 |
| | 48 h | 3.2 ± 1.9 | 0.6 ± 0.5 | >50 |
| | 72 h | 2.9 ± 0.8 | 5.1 ± 2.2 | >50 |
| 6a | 24 h | 1.2 ± 1.1 | 9.9 ± 3.9 | >50 |
| | 48 h | 1.4 ± 0.7 | 3.3 ± 0.3 | 10.1 ± 5.2 |
| | 72 h | 0.5 ± 0.5 | 2.4 ± 0.4 | 6.3 ± 0.7 |
| ASA | 24 h | >50 | >50 | >50 |
| | 48 h | >50 | >50 | >50 |
| | 72 h | >50 | >50 | >50 |
| Inn | 24 h | >50 | >50 | >50 |
| | 48 h | >50 | >50 | >50 |
| | 72 h | >50 | >50 | >50 |
| Sulin | 24 h | >50 | >50 | >50 |
| | 48 h | >50 | >50 | >50 |
| | 72 h | >50 | >50 | >50 |

Data represent the mean ± SD values for cell viability expressed as %, determined by the MTT assay in triplicates.
a GI₅₀: Concentration that reduces by 50% the growth of treated cells compared with untreated controls.
b TGI: Concentration that completely inhibits the cell growth.
c LC₅₀: Concentration that kills 50% of the initial cells.

These results demonstrated that the enhanced cytotoxicity achieved on CRC cells may not simply be the result of incorporating the methylseleno functionality into NSAIDs, but to a unique combination of the methylseleno with ASA, Inn or Sulin. Based on this observation, we generated 6 new ethylseleno and benzylseleno analogs of ASA, Inn, and Sulin (1b, 1c, 5b, 5c, 6b and 6c) and screened them against the same four CRC cells. An overview analysis of the IC₅₀ values obtained and summarized in Table 4 evinced that these six new analogs presented lower antiproliferative activity compared with methylseleno-NSAIDs (1a, 5a and 6a) at 24 hours. Thus, these effects could depend, at least partially, upon the releasing of CH₃SeH or some volatile metabolite. However, the effect of these new derivatives deepened at longer treatments (48 and 72 h), coming to equalize IC₅₀ values of methylseleno-NSAIDs. Surprisingly, the ethylseleno-NSAIDs (1b, 5b and 6b) had no effect on CACO-2 viability (Table 4).

TABLE 4
IC₅₀ values (μM) of 1b, 5b, 6b, 1c, 5c and 6c derivatives and
their analogs 1a, 5a and 6a in different CRC cells.

| | | Time | Cell line | | | |
|---|---|---|---|---|---|---|
| | Comp. | (h) | HT-29 | HCT-116 | RKO | CACO-2 |
| Seleno- | 1b | 24 | >50 | >50 | >50 | >50 |
| ethyl | | 48 | 4.2 ± 0.4 | 13.2 ± 2.6 | >50 | >50 |
| | | 72 | 4.2 ± 0.9 | 6.8 ± 4.2 | >50 | >50 |
| | 5b | 24 | >50 | >50 | >50 | >50 |
| | | 48 | 4.9 ± 0.3 | 6.3 ± 1.6 | 30.8 ± 3.5 | >50 |
| | | 72 | 4.9 ± 0.1 | 8.2 ± 1.7 | 33.3 ± 16.9 | >50 |
| | 6b | 24 | 10.9 ± 1.4 | >50 | >50 | >50 |
| | | 48 | 4.9 ± 0.2 | 6.2 ± 0.9 | 41.8 ± 13.8 | >50 |
| | | 72 | 5.0 ± 0.1 | 7.2 ± 1.0 | 12.4 ± 0.5 | >50 |
| Seleno- | 1c | 24 | >50 | >50 | >50 | >50 |
| benzyl | | 48 | 4.8 ± 0.2 | 8.0 ± 2.1 | 10.4 ± 0.1 | >50 |
| | | 72 | 4.8 ± 0.1 | 8.2 ± 2.2 | 5.0 ± 0.1 | 11.8 ± 5.9 |
| | 5c | 24 | >50 | >50 | >50 | >50 |
| | | 48 | 4.2 ± 0.5 | 14.0 ± 6.6 | 26.2 ± 4.6 | >50 |
| | | 72 | 4.0 ± 1.0 | 5.5 ± 1.2 | 11.5 ± 0.9 | >50 |
| | 6c | 24 | >50 | >50 | >50 | >50 |
| | | 48 | 5.1 ± 0.1 | 7.8 ± 1.8 | >50 | 11.4 ± 1.6 |
| | | 72 | 5.2 ± 0.2 | 7.0 ± 0.8 | 35.8 ± 5.9 | 14.2 ± 3.9 |
| Seleno- | 1a | 24 | 1.9 ± 0.6 | >50 | >50 | 2.6 ± 0.2 |
| methyl | | 48 | 1.2 ± 0.1 | 2.2 ± 0.3 | >50 | 0.9 ± 0.1 |
| | | 72 | 1.2 ± 0.4 | 1. ± 0.2 | >50 | 0.8 ± 0.1 |
| | 5a | 24 | >50 | >50 | >50 | >50 |
| | | 48 | 3.3 ± 0.9 | 5.6 ± 2.6 | 10.1 ± 6.9 | 4.2 ± 1.7 |
| | | 72 | 3.3 ± 0.3 | 18.7 ± 4.9 | 11.6 ± 5.9 | 5.5 ± 1.1 |
| | 6a | 24 | 2.4 ± 0.3 | 7.3 ± 2.3 | >50 | 3.0 ± 0.5 |
| | | 48 | 2.0 ± 0.7 | 3.2 ± 0.7 | 17.2 ± 2.4 | 1.3 ± 0.6 |
| | | 72 | 1.4 ± 0.3 | 1.8 ± 0.6 | 17.3 ± 0.8 | 0.7 ± 0.2 |

Data represent the mean IC₅₀ values (±SD) of % cell viability determined by the MTT assay in triplicate.

Evaluation of the antiproliferative activity of methylseleno-NSAIDs, MSA and NSAIDs alone or in combination. Methylseleno-NSAIDs (1a, 5a and 6a) and the corresponding parent NSAIDs (ASA, Inn and Sulin, respectively) and MSA, alone or in combination, were examined in vitro against a panel of four CRC cells (HT-29, HCT-116, RKO and Caco-2). CRC cells were treated for 24, 48 and 72 h at different concentrations to stablish the dose-response curves.

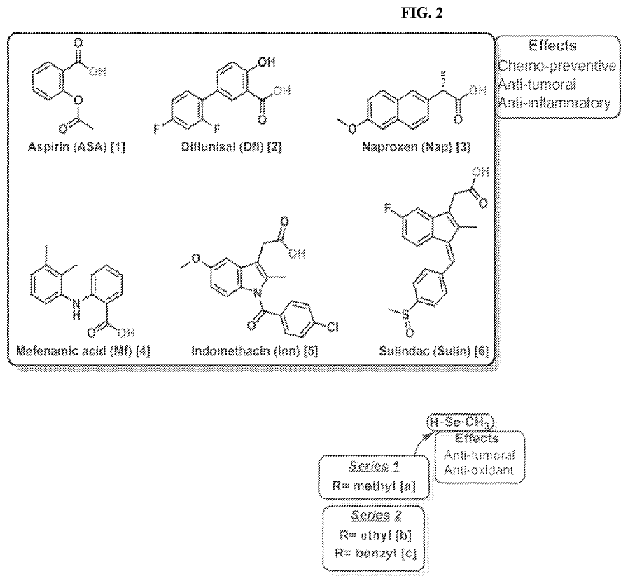

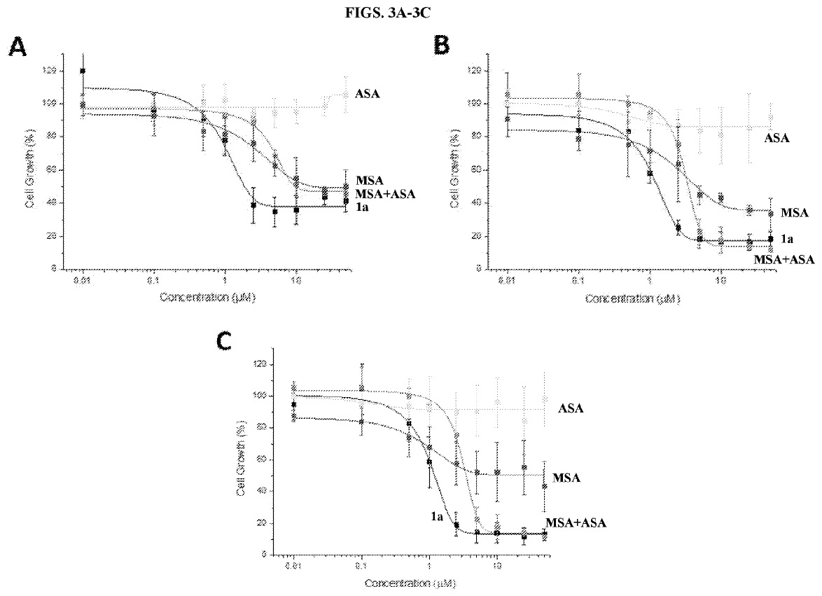

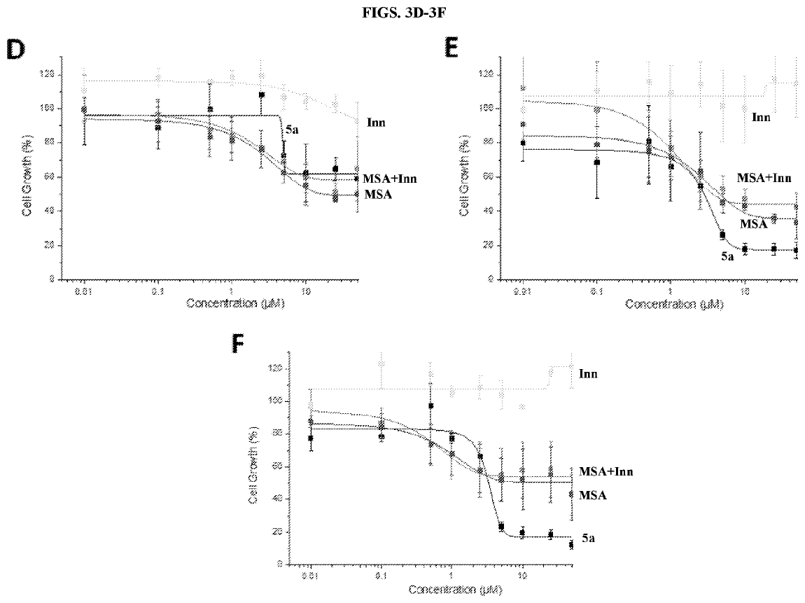

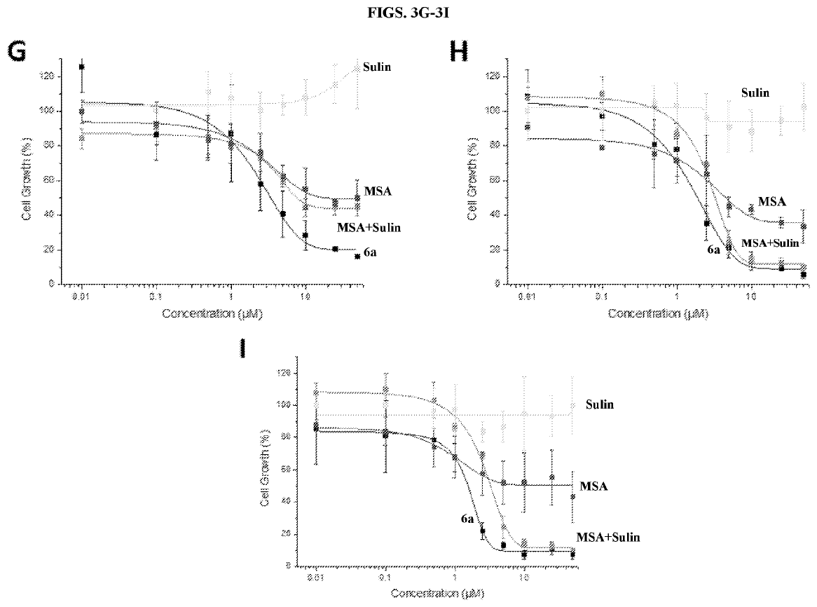

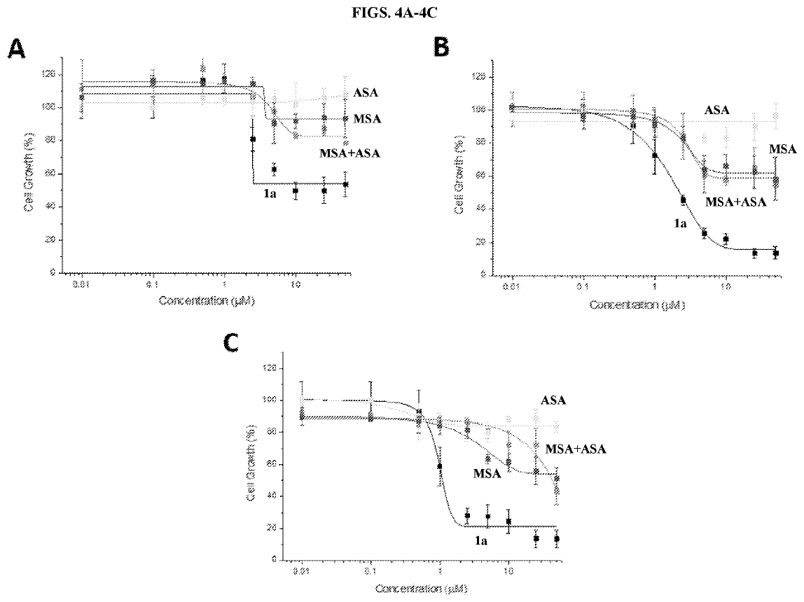

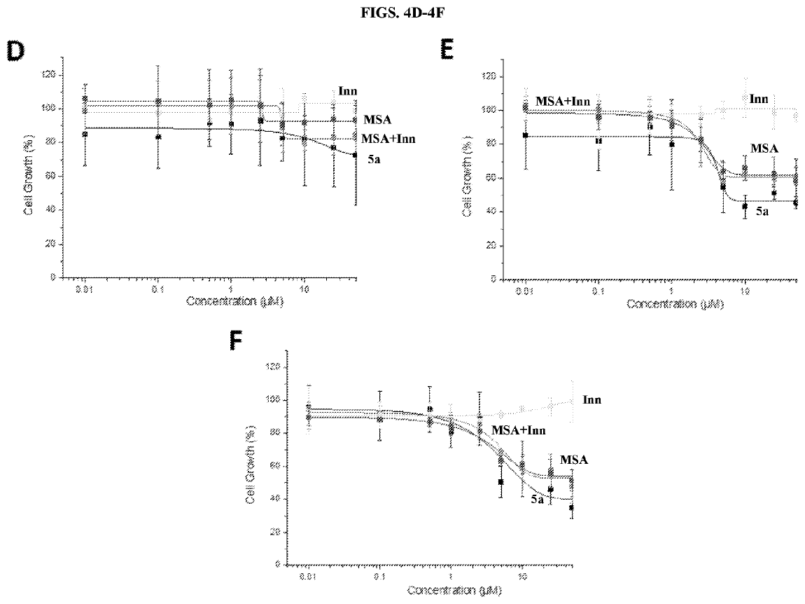

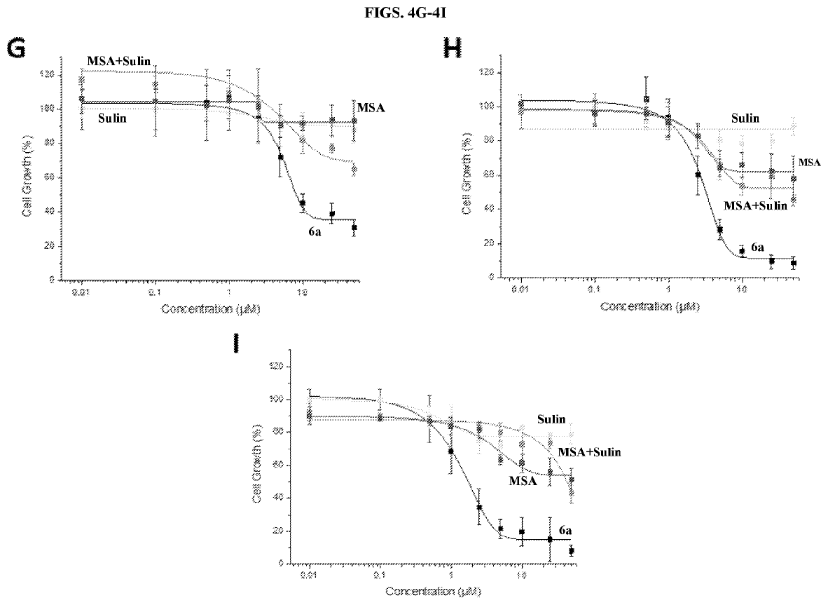

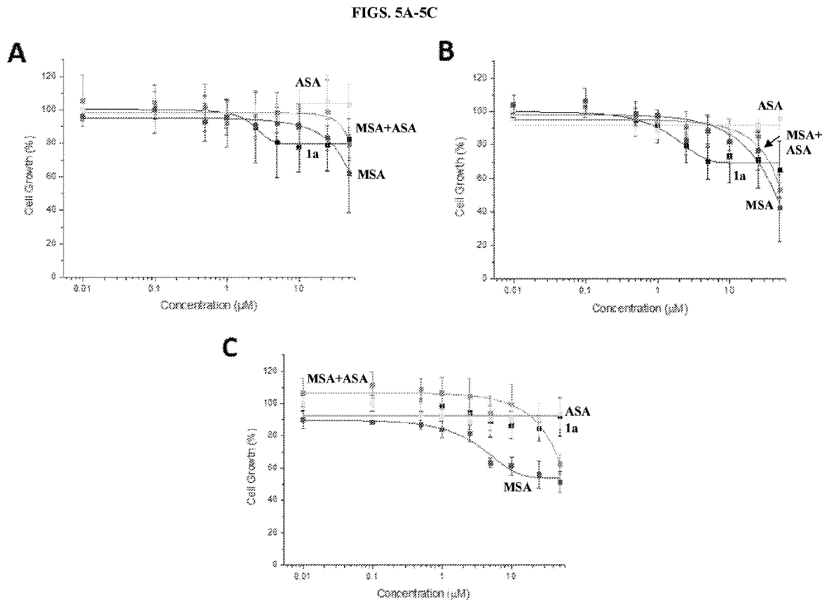

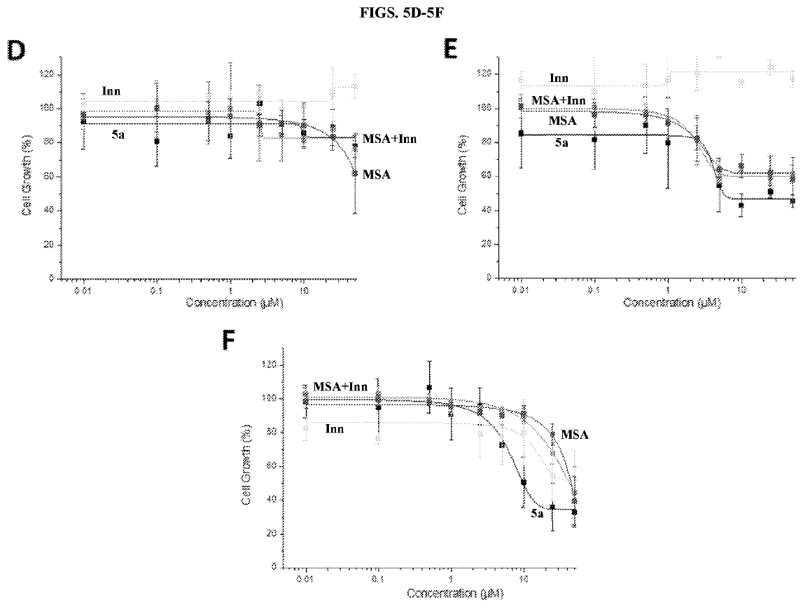

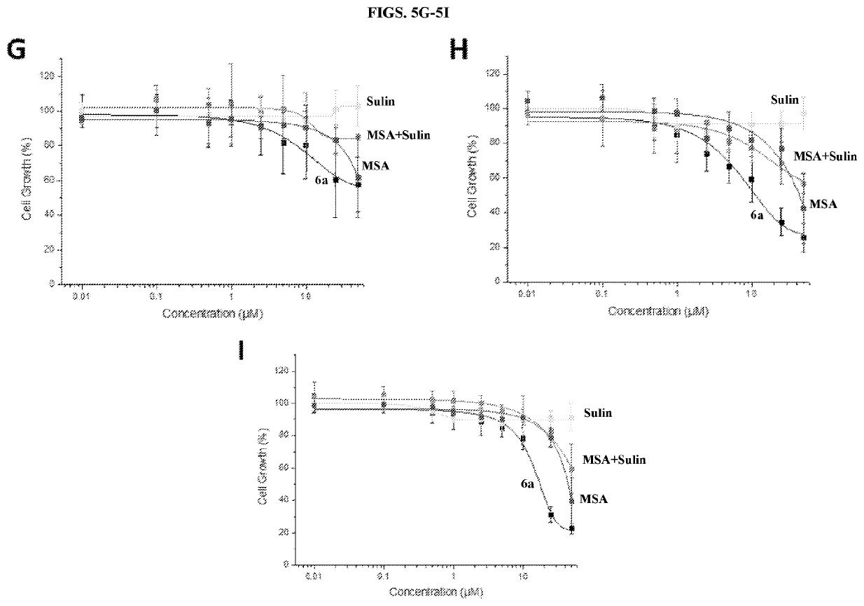

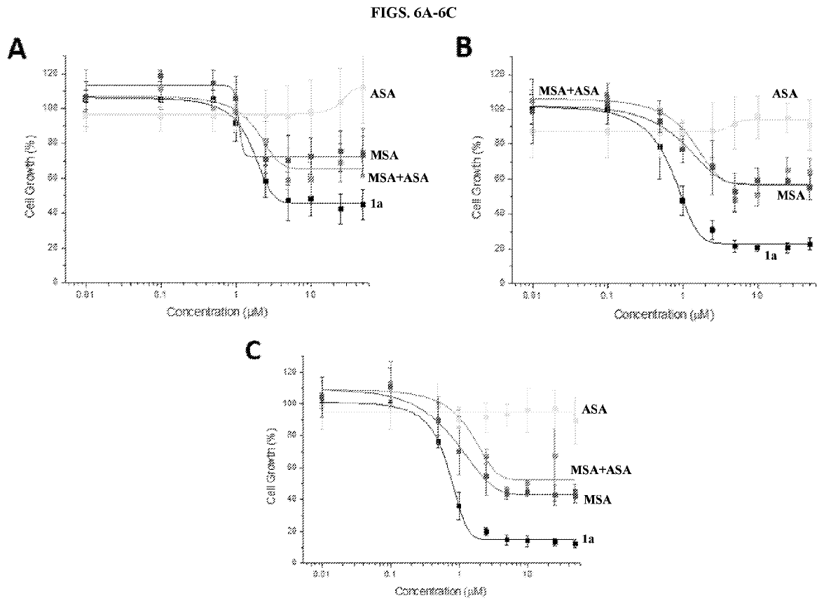

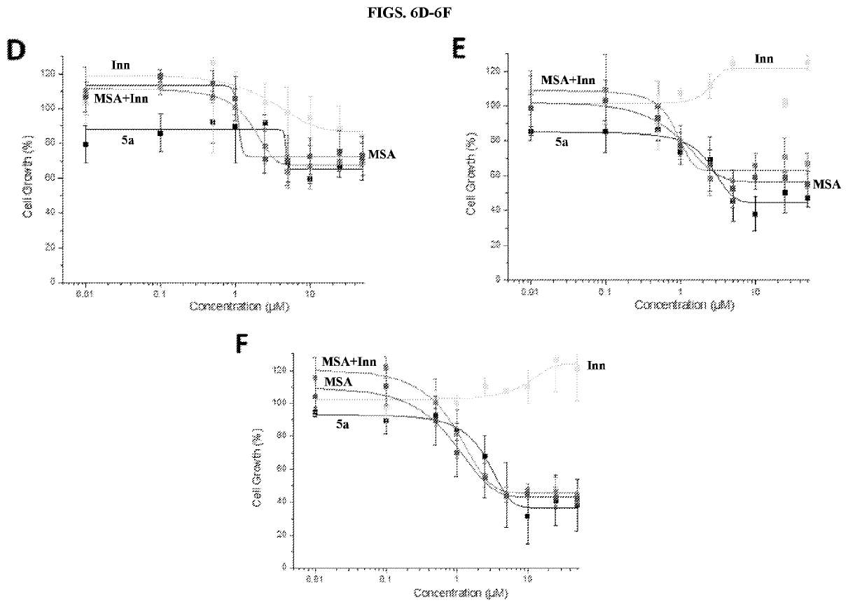

As previously mentioned, MSA is a well-known cytotoxic agent. Interestingly, the results showed synergistic effects of MSA with ASA or Sulin only in HT-29. Nonetheless, MSA combined with Inn did not show synergistic effects (FIGS. 3A-3I). In addition, the assembly of precursor of CH₃SeH and NSAIDs in the same molecule (1a, 5a and 6a) demonstrated higher anti-tumoral activity in the 4 CRC cells used than MSA and NSAIDs (ASP, Inn and Sulin, respectively) as single agents or combined (FIGS. 3-6). Thus, evidences suggest these methylseleno-NSAIDs can be considered very promising candidates to become chemo-therapeutic drugs.

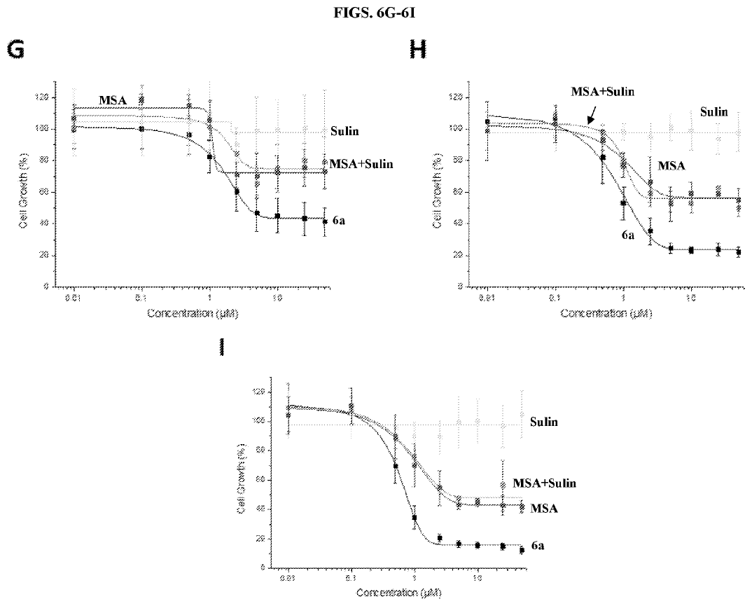

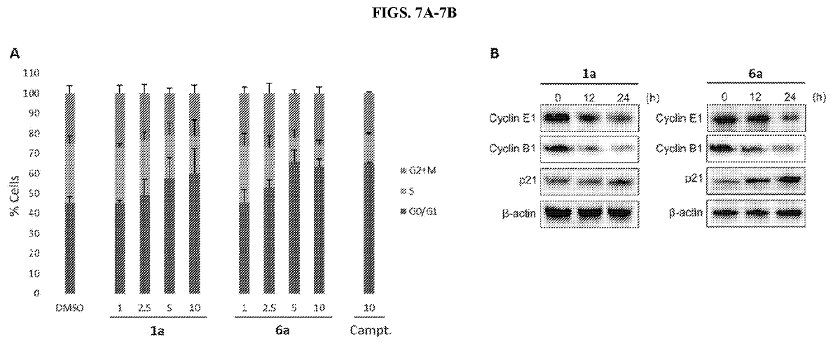

Compounds 1a and 6a inhibited CRC cell growth by inducing cell cycle arrest. We investigated the effect 1a and 6a on the cell cycle by flow cytometry. Treatment of HT-29 cells with both 1a and 6a demonstrated an increase in the large population of cells in G1 and G2/M phases of the cell cycle compared with vehicle (DMSO) after 24 h of treatment. The population of cells in the S-phase was drastically reduced after the treatment with compounds 1a and 6a compared with vehicle (FIG. 7A).

To further explore the underlying mechanism of the cell cycle arrest, we studied the expression levels of the different proteins involved in the cell cycle regulation. After the treatment with compounds 1a and 6a, the expression levels of proteins Cyclin B1 (required for cells to enter the mitosis phase) and Cyclin E1 (required for cells to transit from G1 to the S phase) went down substantially. However, a sharp increase in the expression level the cyclin dependent kinase inhibitor p21 was observed, which has been associated with G1/S and G2/M cell cycle arrest. The cell cycle inhibition effect of compounds 1a and 6a was observed at two different time intervals using IC₅₀ dose of the compounds. At both time intervals of 12 h and 24 h, the expression levels of cell cycle regulatory proteins cyclin E1 and cyclin B1 were inhibited while p21 was induced (FIG. 7B).

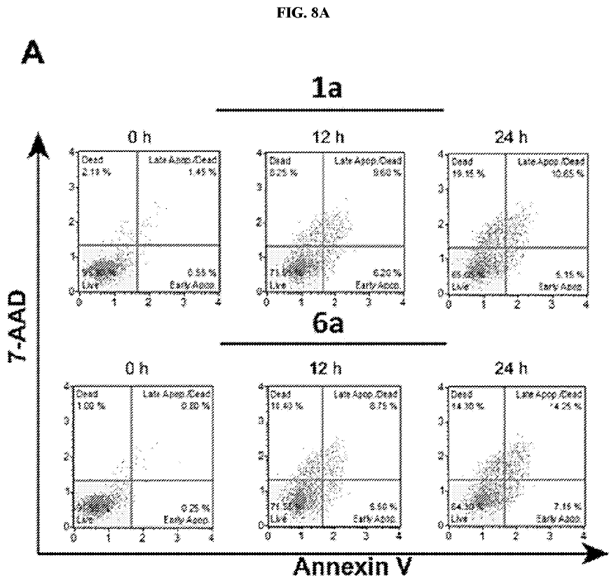

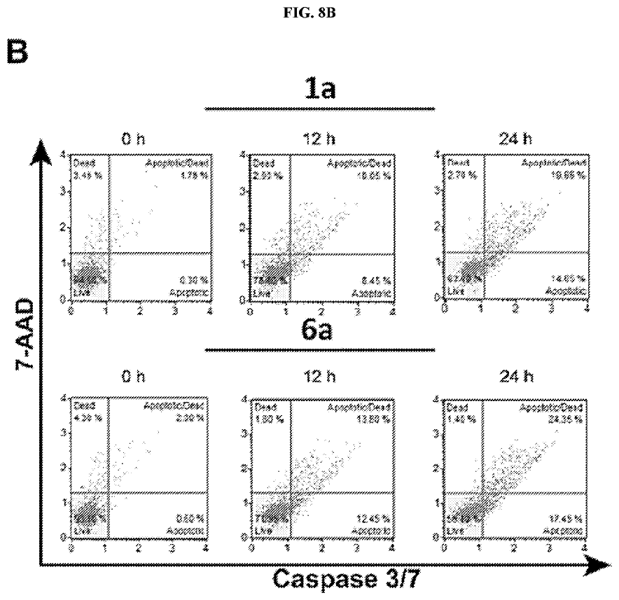

Compounds 1a and 6a induced apoptosis in CRC cells. We further investigated whether the inhibition of cell cycle leads to apoptosis. In pursuit of this, HT29 cells were treated with 5 μM of the compounds 1a and 6a for indicated time intervals. Apoptosis was measured by using Annexin V & Dead cell assay kit and Caspase 3/7 Assay kit using Muse cell analyzer following manufacturer's protocol. The live cell population resided in the lower left quadrant and they start shifting towards the lower right quadrant (early apoptotic) or to upper right quadrant (late apoptotic/dead) after treatments. At 5 μM concentration of both compounds for 12 hours, compound 1a showed <76% live cells, and after 24 h time, the live cells population further decreased to <65%. A similar trend was also observed after 6a treatment where the live cell population were decreased from <75% to <65% after 12 h and 24 h treatment, respectively. Similarly, the Caspase 3/7 Assay analysis showed time dependent increase in the apoptotic cell population after treatment with the compounds 1a and 6a (FIG. 8).

NCI-60 analysis. Compounds 1a, 5a and 6a were submitted to the National Cancer Institute's (NCI) Developmental Therapeutics Program (DTP). NCI-60 program consists in a panel of 60 human cancer cell lines from a variety of cancers, including refractory tumors such as lung, ovarian, CRC, breast, brain and renal cancers, and melanomas together with more treatable cancers such as leukemias (71). 1a [NSC: 803071], 5a [NSC: 820332] and 6a [NSC: 811013] were initially screened at one dose (10 μM) at 48 h. Then, NCI selected only compound 6a to perform dose-response characterization (72).

As shown in Figure S1 (Exhibit A), compound 1a at 10 μM was highly selective, only presenting significant cytostatic activity (with Growth percent (GP)<12%), in 3 out of the 60 tumor cell lines tested (HT-29 (CRC cells), RXF-393 (renal cancer) and NCI-H226 (non-small cell lung cancer)). Interestingly, NCI-H226 is among the ten most resistant cancer cell lines in the NCI-60 panel. In addition, this derivative has shown cytotoxic activity only in a central nervous system (CNS) cancer cell (RXF-393), with GP values of −42.41%.

Compound 5a at 10 μM demonstrated potent antitumor activity in most NCI-60 cell lines, with a GP mean value towards all cell lines of 4.01% (Figure S2 in Exhibit A). Remarkably, analogs 5a showed great cytotoxic activity in some of the most resistant cancer cells of NCI panel towards more than 20,000 compounds tested, i.e., melanoma (SK-MEL-28, GP equal to −7.85%), prostate (DU-145, GP equal to −7.90%), renal (TK-10, GP equal to −58.40%) and breast (BT-549, GP equal to −80.79%) (71).

Remarkably, compound 6a, the derivative of Sulin, presents impressive cytotoxic activity, with GI50, TGI and LD50 values in the nanomolar range in several lung, CNS, melanoma and renal cancer cell lines (Table 5). GI50 values below 10 nM were found in four NSCLS and two CNS cell lines and TGI values were below 12 μM in all cell lines, except for seven, four of them being leukemia cells (Figure S2, Supporting Information in Exhibit A). Besides, 6a is a cytostatic agent towards all the 6 cell lines derived from breast cancer, but it showed a selective profile against MDA-MB-231/ATCC.

TABLE 5
Cell growth inhibition parameters of 6a derivative
on NSCLC, CNS, melanoma and renal cancers at 48 h.

| | GI₅₀^{a} | TGI^{b} | LC₅₀^{c} |
|---|---|---|---|
| Cell line | (nM) | (nM) | (nM) |
| ME: LOX IMVI | 14.1 | 29.6 | 62.1 |
| CNS: SF-295 | 24.2 | 107 | 386 |
| RE: A498 | 38.8 | 184 | 658 |
| CNS: SF-539 | 105 | 275 | 718 |
| LC: EKVX | <10 | 10.8 | 861 |
| LC: HOP-62 | <10 | 155 | 941 |

^{a}GI₅₀: Concentration that reduces by 50% the growth of treated cells with respect to untreated controls.
^{b}TGI: Concentration that completely inhibits the cell growth.
^{c}LC₅₀: Concentration that kills 50% of the initial cells.

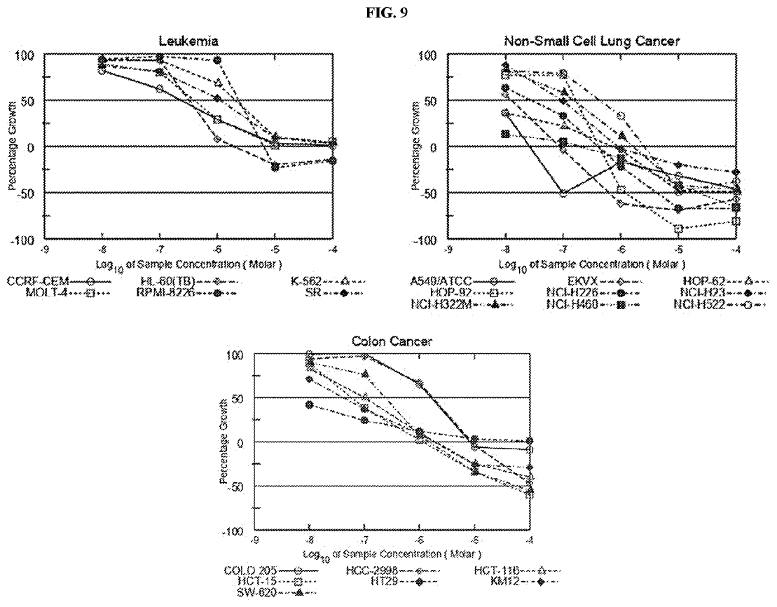

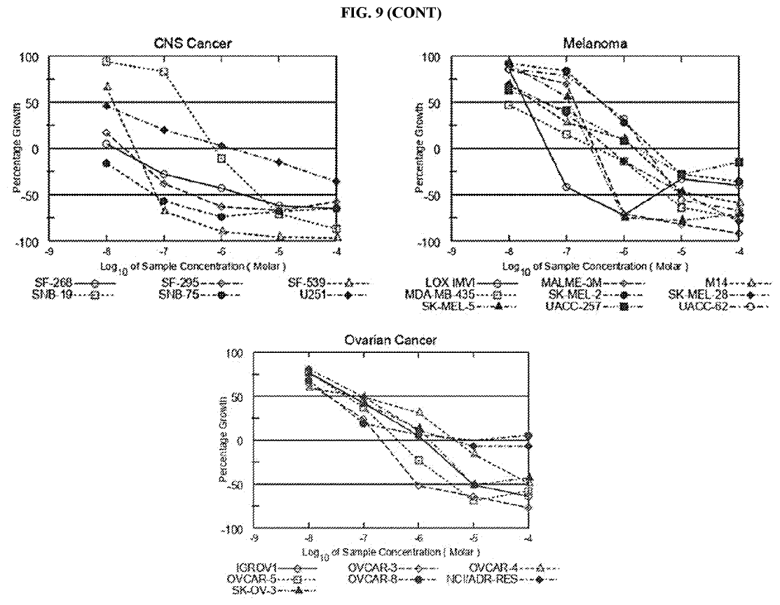

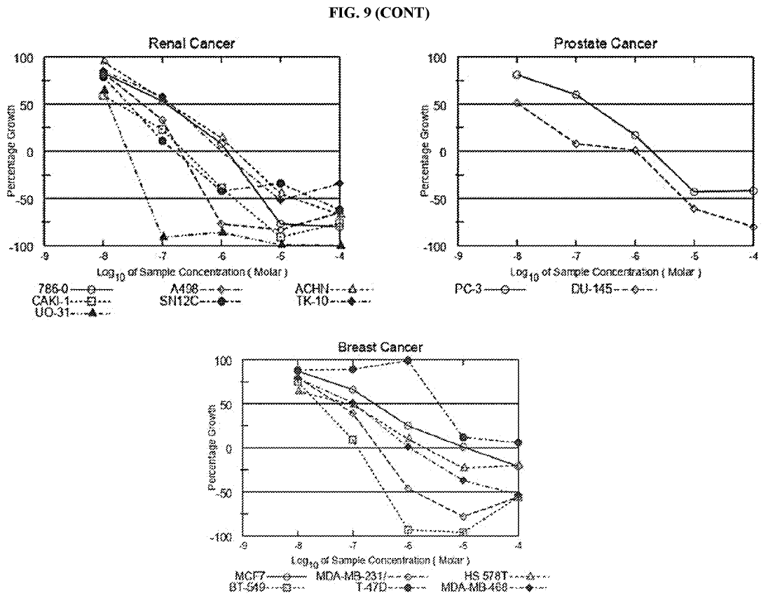

A comparison with the chemical database of over 20,000 compounds evaluated by the NCI, showed that the cytotoxic activity of 6a was negligible towards the 6 most sensitive cells of the panel (LE:SR, LE: CCRF-CEM, CO: HCT-116, LE: HL60, LC: NCI-H522 and LE: MOLT-4), presenting on the contrary outstanding activity towards the 6 most resistant cells (RE: TK10, ME: SK-MEL-28, CNS: SNB-19, OV: SK-OV-3, LC: NCI-H322M, OV: OVCAR-5), with cytotoxic parameter values of 47, 410 and 3900 nM for GI50, TGI and LC50, respectively, in the most resistant cell line (OVCAR-5) (FIG. 9) (71).

The GI50, TGI and LC50 values of 6a [NSC: 811013] were analyzed using the COMPARE algorithm. This algorithm provides an automated and quantitative comparison of the cytotoxic profile newly designed compounds with the patterns of NCI-60 chemical databases, including standard clinical drugs, in order to delineate potential mechanisms of actions (73). The comparison results are represented as Pearson's correlation coefficients (r) ranging from −1 to +1, +1 meaning high degree of homology of cytotoxic profile. Interestingly, this similarity study using the COMPARE revealed an unprecedented behavior of 6a, since this analog only match some similarity with romidepsin, but the correlation factor was below 0.5. Romidepsin, also known as Istodax, is an antitumor drug used in cutaneous and peripheral T-cell lymphomas. It has shown acceptable toxicity profile and clinically meaningful efficacy in patients with peripheral T-cell lymphoma in phase I/II clinical trials (74, 75). This anticancer drug works by blocking histone deacetylases, which results in alterations in gene expression and the induction of cell differentiation, cell cycle arrest and apoptosis (76, 77). Interestingly, this drug increased the antiproliferative and proapoptotic effects of gemcitabine both in vitro and in vivo pancreatic ductal adenocarcinoma models (78). Romidepsin is currently in clinical stages for pancreatic cancer treatment [NCT04257448] [NCT00379639] (79).

Conclusions

In conclusion, the addition of methylseleno, ethylseleno and benzylseleno fragments into NSAIDs led to the discovery of novel analogs as potential cancer therapeutics. Among this new series of 12 seleno NSAID analogs, three methylseleno-NSAIDs (1a, 5a and 6a) exhibited greater cytotoxic activity compared with methylselenilic acid and parent NSAID scaffolds (ASA, Inn and Sulin, respectively) alone or in combination in CRC cells. In addition, our studies revealed that 1a and 6a inhibited HT-29 growth via cell cycle arrest leading to apoptosis. In addition, 1a and 6a induced apoptotic cell death evident by caspase 3/7 activity. Furthermore, analogs 1a, 5a and 6a were then submitted to NCI DTP to evaluate their activity in a panel of 60 cancer cell lines. These analogs showed completely different profiles. Whilst analog 1a only presented negative growth inhibition in one central nervous system (CNS) cell line (RXF-393), with a growth percent of −42,41, analog 5a demonstrated great cytotoxic activity in the most resistant melanoma (SK-MEL-28), prostate (DU-145), renal (TK-10) and breast (BT-549) cancer cells of NCI panel towards more than 20,000 compounds tested. On the other hand, analog 6a demonstrated striking cytotoxic activity with GI50, TGI and LD50 values in the nanomolar range in several lung, CNS, melanoma and renal cancer cell lines. Outstandingly, 6a possessed low activity towards the 6 most sensitive cells and potent cytotoxicity against the 6 most resistant cells of the DTP panel, with cytotoxic parameter values of 47, 410 and 3900 nM for GI50, TGI and LC50, respectively, in the most resistant cell line (OVCAR-5). Surprisingly, a similarity study using the COMPARE algorithm unveiled an unprecedented behavior for 6a, since this analog only match minimal similarity with romidepsin. To conclude, methylseleno-NSAID 6a showed unique profile with demonstrated in vitro and in vivo antitumor efficacy and can be a potential drug-candidate for further development. Future studies warrant the detailed study of efficacy and mechanism of action of lead analog 6a.

### Example 2—In Vitro and In Vivo Antitumor Efficacy Studies in Pancreatic Cancer

The methylseleno-sulindac analog 6a showed a unique behavior in that it was effective against six most resistant cell lines in the NCI-60 cancer cell line panel that includes leukemia, breast, colon, CNS, prostate, melanoma, renal, non-small cell lung and ovarian cancer cell lines.

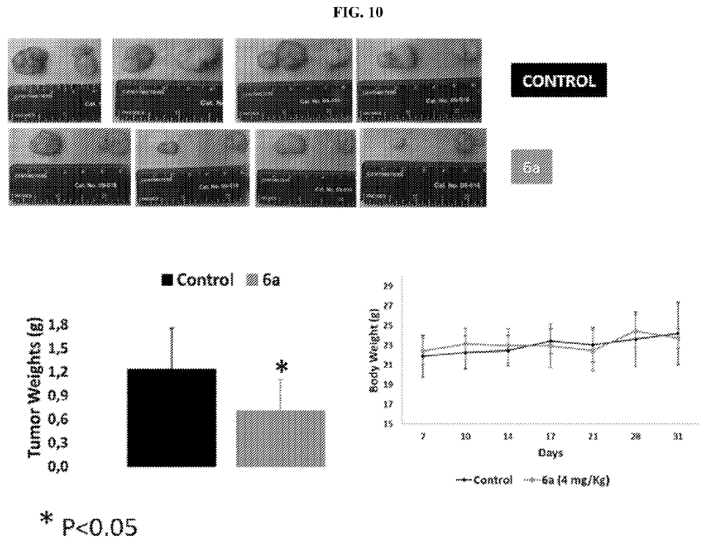

The cytotoxic response of 6a on pancreatic ductal adenocarcinoma cell lines (Panc-1, MIA PaCa-2 and BxPC3) was evaluated using MTT assay. Our data shows that 6a was most effective on BxPC3 cells and inhibited their viability on, with an IC50 value of 1 μM. To determine whether the in vitro efficacy of 6a translates to in vivo models, we performed a pilot tumor inhibition study in xenograft mouse model using BxPC3 cells. Athymic nude mice were subcutaneously injected with BxPC3 cells. After two weeks, mice were injected intraperitoneally with vehicle DMSO (control) or 6a (three times a week at a dose of 4 mg/Kg in 100 μL of DMSO). Tumor size and animal weights were measured twice a week. As shown in FIG. 10, 6a significantly inhibited tumor growth and by the end of the experiment (22 days since start of the treatment), 6a reduced tumor weight by 42.9% as compared to control mice. Additionally, no evidence of systemic toxicity was observed at the dose used as evidenced by the similar liver basal levels to control for the following parameters: 23 mg/dl of blood urea nitrogen, 0.40 mg/dl of bilirubin toxicity, 105 U/L aspartate aminotransferase (SGPT) and 19 U/L alanine aminotransferase (SGPT). Besides, body weight loss is often used as a measure of drug induced toxicity, 6a-treated mice did not show any significant weight loss as compared to the control mice. Hence, tumor inhibition along with in vitro results and low toxicity strongly suggest the potential of 6a as a novel agent for the treatment of pancreatic cancer.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

We claim:

1. A compound of formula (I), or a pharmaceutically acceptable salt thereof: 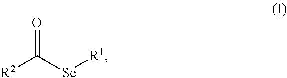
wherein R¹ is selected from alkyl and (aryl)alkyl;
wherein R² is selected from aryl, (aryl)alkyl, heteroaryl, and (heteroaryl)alkyl, and R² optionally is substituted with one or more R^{A}; and
wherein R^{A} is selected from alkyl, alkoxy, —O—C(O)H, —O—C(O)—CH₃, halogen, phenyl optionally substituted with one or more halogens, N-anilino optionally substituted with alkyl, —C(O)-phenyl optionally substituted with halogen, —CH═CH-phenyl optionally substituted with —S(O)—CH₃.

2. The compound of claim 1, wherein R¹ is alkyl.

3. The compound of claim 1, wherein R¹ is methyl or ethyl.

4. The compound of claim 1, wherein R¹ is (aryl)alkyl.

5. The compound of claim 1, wherein R¹ is benzyl.

6. The compound of claim 1, wherein R² is selected from phenyl, -alkyl-naphthalene, -alkyl-indolyl, and -alkyl-indenyl, optionally substituted with one or more R^{A} groups.

7. The compound of claim 1, wherein R² is phenyl optionally substituted with one or more R^{A}.

8. The compound of claim 1, wherein R² has a formula selected from

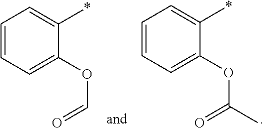 [SMILES: *c1ccccc1OC=O ; *c1ccccc1OC(C)=O]

9. The compound of claim 1, wherein R² has a formula

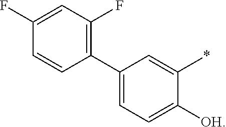 [SMILES: *c1cc(-c2ccc(F)cc2F)ccc1O]

10. The compound of claim 1, wherein R² has a formula

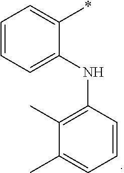 [SMILES: *c1ccccc1Nc1cccc(C)c1C]

11. The compound of claim 1, wherein R² is -methyl-naphth-2-yl optionally substituted with one or more R^{A}.

12. The compound of claim 1, wherein R² has a formula

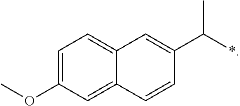

13. The compound of claim 1, wherein R² is -methyl-1H-indol-3-yl optionally substituted with one or more R^{A}.

14. The compound of claim 1, wherein R² has a formula

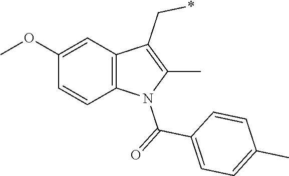

15. The compound of claim 1, wherein R² is -methyl-1H-inden-3-yl optionally substituted with one or more R^{A}.

16. The compound of claim 1, wherein R² is has a formula

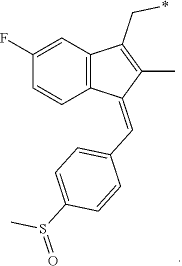 [SMILES: *CC1=C(C)/C(=C/c2ccc(S(C)=O)cc2)c2ccc(F)cc21]

17. The compound of claim 1 having a formula selected from:

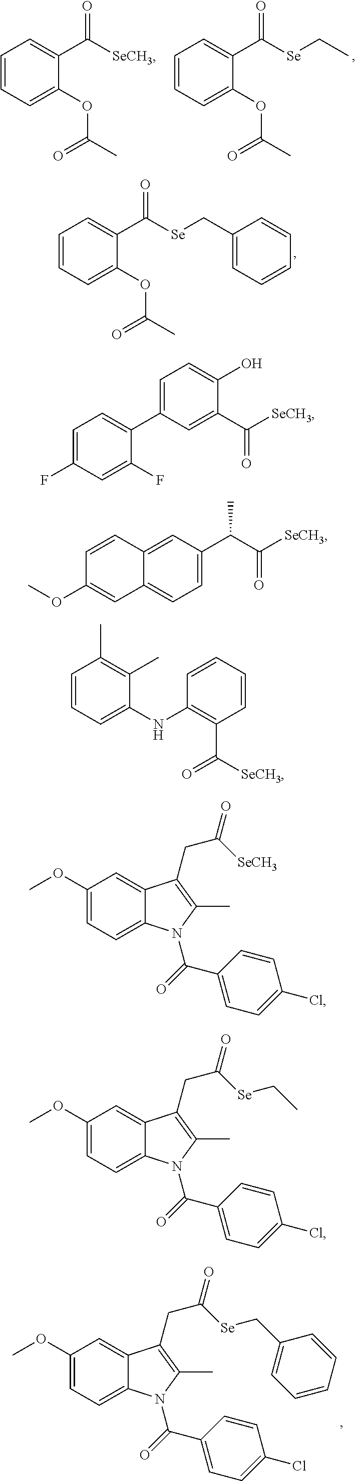

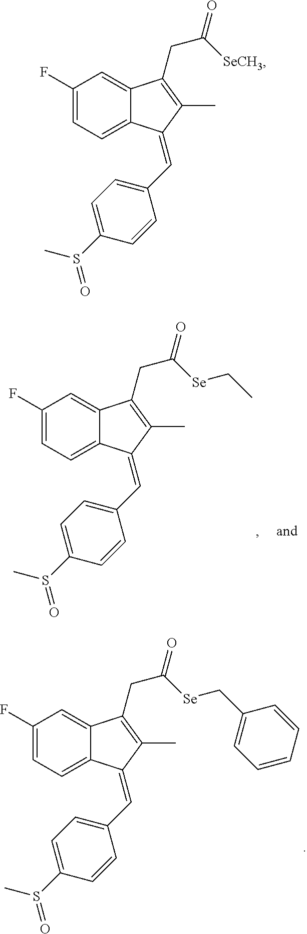

18. A pharmaceutical composition comprising the compound of claim 1 and a pharmaceutically acceptable carrier.

19. A method of treating cancer in a subject in need thereof, the method comprising administering to the subject the compound or pharmaceutically acceptable salt of claim 1 in a therapeutically effective amount to treat the cancer.

20. The method of claim 19, wherein the cancer is selected from the group consisting of pancreatic ductal adenocarcinoma, melanoma, prostate cancer, breast cancer, renal cancer, and any combination thereof.
